(19) United States
(12) Patent Application Publication
(10) Pub. No.: US 2003/0073721 A1
(43) Pub. Date: Apr. 17, 2003

(54) Peptide beta-strand mimics based on 1,2-dihydro-3(6H)-pyridinone

(75) Inventors: Paul A. Bartlett, Oakland, CA (US); Miroslav Rezac, Chicago, IL (US); Steven Olson, Metuchen, NJ (US); Scott Phillips, Berkeley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, a California corporation, Oakland, CA (US)

(21) Appl. No.: 10/157,759

(22) Filed: May 28, 2002

Related U.S. Application Data
(60) Provisional application No. 60/296,167, filed on Jun. 5, 2001.

(51) Int. Cl. C 07D 4 1/14; C 07D 4 1/02; A61K 31/444; A61K 31/4439; A61K 31/44

(52) U.S. Cl. 514/333; 514/335; 514/350; 514/341; 514/339; 546/256; 546/261; 546/272.7; 546/277.4; 546/276.4; 546/298

(57) ABSTRACT

Peptide analogs formed by replacing one or more, but not all, amino acids of a peptide chain with 1,2-dihydro-3(6H)-pyridinone, display an unusually strong tendency to assume a β-strand conformation and to enter into β-sheet-like interactions with peptides and other peptide analogs that engage in β-sheet-like interactions with peptides. The peptide analogs of this invention therefore have utility has β-strand mimics offering advantages over native peptides as well as β-strand mimics of the prior art.

### CROSS-REFERENCE TO RELATED APPLICATION

This application claims the benefit of co-pending U.S. provisional patent application No. 60/296,167, filed Jun. 5, 2001, for all purposes legally served thereby. The contents of provisional patent application No. 60/296,167 are incorporated herein by reference in their entirety. All other patent and literature references cited throughout this specification are likewise incorporated herein by reference in their entirety.

### STATEMENT AS TO RIGHTS TO INVENTIONS MADE UNDER FEDERALLY SPONSORED RESEARCH AND DEVELOPMENT

This invention was made with Government support under Grant (Contract) Nos. GM30759 and AG10770 awarded by the National Institutes of Health. The Government has certain rights to this invention.

### BACKGROUND OF THE INVENTION

1. Field of the Invention

This invention resides in the field of proteins and the complexations and interactions of proteins with other proteins and with nucleic acids through β-sheet interactions. The particular areas addressed by this invention are compositions for and methods of modifying the ability of proteins to enter into these interactions and the various benefits that are derived from such modifications, including changes to the biological activity of the proteins.

2. Description of the Prior Art

The conformation of proteins and peptides is largely governed by secondary structural elements, such as α-helices, β-turns, and β-strands, which determine the three-dimensional orientation of the amino acid side chains and thereby the longer range interstrand and intermolecular interactions. Both β-strands and the β-sheets derived from β-strands play important roles in protein-protein interactions as well as the association of proteins with other biopolymers such as ribosomal RNA and nucleic acids. Disclosures of these roles are found in Fitzgerald, F. M. D., et al., J. Biol. Chem. 1990, vol. 265, 14209; Zutshi, R., et al., J. Am. Chem. Soc. 1997, vol. 119, 4841; Babe, L. M., et al., Protein Sci. 1992, vol. 1, 1244; Siligardi, G., et al., Biopolymers (peptide science) 1995, vol. 37, 281; Stanfield, R. L., et al., Current Opinion in Structural Biology 1995, vol. 5, 103; Buckle, A. M., et al., Proc. Natl. Acad. Sci. USA 1997, vol. 94, 3571; Taneja, B. C., et al., Protein Engineering 1999, vol. 12, 815; Stern, L. J., et al., Nature 1994, vol. 368, 215; Moss, N., et al., J. Med. Chem. 1996, vol. 39, 2178; Sauer, F. G., et al., Science 1999, vol. 285, 1058; and Karlsson, K. F., et al., J. Bioorg. Med. Chem. 1998, vol. 6, 2085. For example, the β-sheet-like association and precipitation of hydrophobic protein fragments in amyloid plaques is strongly implicated in neurodegenerative diseases, as disclosed by Roloff, E. V., et al., Cell. Mol. Life Sci. 1999, vol. 55, 601; Yatin, S. M., et al., J. Mol. Neurosci. 1998, vol. 11, 183; and Prusiner, S. B., et al., Cell 1998, vol. 93, 337. Furthermore, various biological processes depend on the accessibility of individual peptide strands. Examples of these processes are:

vancomycin complexation of the Lys-D-Ala-D-Ala peptide in bacterial cell wall synthesis;

homodimerization of HIV protease, which involves a “fireman's grip” β-sheet interaction among the N-terminal residues;

heterodimerization of ribonucleotide reductase and HIV reverse transcriptase, which can be blocked with soluble oligopeptides corresponding to part of the interface regions;

dimerization of the γ-Cro repressor via an antiparallel β-strand; and

protein-protein association via PDZ domains.

Systems that mimic and block these interactions are disclosed by Smith, A. B., et al., J. Am. Chem. Soc. 1992, vol. 114, 10672; Smith, A. B., et al., J. Am. Chem. Soc. 1994, vol. 116, 9947-9962; Smith, A. B., et al., J. Am. Chem. Soc. 1995, vol. 117, 11113-11123; Smith, A. B., et al., Bioorg. Med. Chem. 1996, vol. 4, 1021; Smith, A. B., et al., J. Am. Chem. Soc. 1999, vol. 121, 9286-9298; Smith, A. B., et al., Organic Letters 2000, vol. 2, 2037; Smith, A. B., et al., Organic Letters 2000, vol. 2, 2041; Hirschmann, R., et al, U.S. Pat. No. 5,489,692, issued Feb. 6, 1996; Hirschmann, R. F., et al, U.S. Pat. No. 5,514,814, issued May 7, 1996; Hirschmann, R. F., et al, U.S. Pat. No. 5,770,732, issued Jun. 23, 1998; Smith, III, A. B., et al, U.S. Pat. No. 6,034,247, issued Mar. 7, 2000; Nowick, J. S., et al., J. Am. Chem. Soc. 2000, vol. 122, 654-661; Nowick, J. S., et al., J. Am. Chem. Soc. 2001, vol. 123, 5176-5180; Nowick, J., et al., International Patent Application No. WO 01/14412, published Mar. 1, 2001, under the Patent Cooperation Treaty; and Kemp, D. S., et al., J. Org. Chem. 1990, vol. 55,4650-7.

Among these disclosures, those of Smith and Hirschmann involve the use of pyrrolinone rings in oligomers whose subunits are linked by carbon-carbon bonds, each monomer in the oligomer having a different side chain and thereby requiring a separate synthesis. Those of Nowick involve the use of hydrazides, aromatic acids, and oxamides of aromatic acids. The disclosure of Kemp et al. involves a tetracyclic epidolindione derivative as a non-repeating template.

### SUMMARY OF THE INVENTION

It has now been discovered that 1,2-dihydro-3(6H)-pyridinones, referred to herein for convenience as “azacyclohexenones” or “Ach” units, are unusually effective as units in peptide β-strand mimics, i.e., as amino acid substitutes in peptide analogs, in view of the unique ability of these analogs to assume β-sheet conformations and to engage in intermolecular interactions with peptides as β-sheet templates. Peptide analogs in which at least one but less than all amino acids is replaced by an azacyclohexenone unit of the present invention readily enter into β-sheet-like interactions, and these analogs as well as the azacyclohexenones themselves are simpler to synthesize than the peptide mimics of the prior art.

The azacyclohexenones of this invention thus form peptide analogs, also referred to herein as β-strand mimics, with ordered structures that allow each analog to serve as a template for association with a peptide strand or with the edge of a β-sheet through hydrogen bonding to the backbone amides of the strand or sheet. As with β-sheet-like interactions between naturally occurring peptides, the side-chain interactions between the peptide analog and the peptide provide sequence selectivity.

Also encompassed by this invention are constructs that consist of a conventional peptide sequence covalently linked to a peptide analog sequence in which at least one but not all amino acids is replaced by an azacyclohexenone unit, the linkage being one that permits a β-turn. Such a construct is also referred to herein as a “hybrid” since it contains both a conventional peptide sequence (i.e., one that does not contain an azacyclohexenone unit) and an azacyclohexenone-containing sequence. The azacyclohexenone-containing portion of the construct has a strong tendency to enter into a stable β-sheet-like interaction with the conventional peptide portion, thereby stabilizing the conventional peptide portion in a β-strand conformation that serves as a template for β-sheet-like interactions with other peptides.

The peptide analogs and peptide-analog hybrids of this invention have many applications. They can for example serve as tools for studying β-sheet nucleation, propagation, and suppression. They can also serve as prophylactic or palliative agents in physiological conditions that involve or are controllable by β-sheet interactions. For example, these peptide analogs and hybrids can be used in the treatment of prion diseases such as “mad cow disease” and other neurodegenerative diseases such as Alzheimer's disease which arise from the association of certain hydrophobic proteins to form insoluble β-sheet aggregates known as amyloid complexes. This utility arises from the enhanced ability of the analogs and hybrids of this invention to bind to an exposed surface of the amyloid β-sheet complex and prevent further aggregation. The peptide analogs and hybrids can also be used for blocking the infectivity of the human immunodeficiency virus by inhibiting the association of the viral gp 120 protein with the CD4 receptor on the T-lymphocyte cell surface. A still further use is the blocking of the effects of inflammatory chemokines that are involved in allergic reactions, psoriasis, arthritis, atherosclerosis, multiple sclerosis, and cancer.

Peptide analogs in accordance with this invention can operate in a manner similar to an antibody by binding to peptides and proteins in a sequence-selective manner, such as for example as capture peptides covalently bonded to solid supports. As such, the peptide analogs and peptide-analog hybrids of this invention are useful for example as protein purification media in affinity chromatography. They are also useful as components in diagnostic devices or kits, where they can be used for the concentration and identification of peptide and protein analytes. This antibody-type character also provides utility in vivo, where the peptide analogs and peptide-analog hybrids can be used for therapeutic effects by complexing with and blocking the action of specific peptide hormones or by targeting attached radiopharmaceuticals or cytotoxic agents to specific sites in the body. A collection of peptide analogs and hybrids in accordance with this invention can be arranged in an array such as that of a proteomics chip for use in an assay for the levels of expression of specific proteins in different tissues and under different conditions. Other uses will be readily apparent to those skilled in the art.

The present invention thus resides in:

1,2-Dihydro-3(6H)-pyridinones (“azacyclohexenones”), either functionalized for linkage to each other or to amino acids through carbon-nitrogen (peptide-type) bonds, or covalently bonded to one or more amino acids through peptide-type bonds, as well as peptide analogs in which at least one amino acid, but not all, is replaced by an azacyclohexenone group, and peptide-analog hybrids consisting of peptides covalently linked to peptide analogs through β-turn-permitting linkages, all as compositions of matter

The use of peptide analogs and peptide-analog hybrids as described above for inhibiting β-sheet-like interactions between proteins

The use of peptide analogs and peptide-analog hybrids as described above for inhibiting the biological activity of a peptide

The use of peptide analogs and peptide-analog hybrids as described above for extracting a target peptide from a mixture of peptides

Other aspects, embodiments, applications and features of the invention will be apparent from the description that follows.

### DESCRIPTION OF THE DRAWINGS

FIG. 1 is a plot of the dissociation constant K_{d} of a dimer of a peptide analog in accordance with this invention in a solution in which the solvent is a mixture of CD₃OH and CDCl₃ as a function of the concentration of CD₃OH in the solvent mixture.

FIGS. 2a and 2b are molecular diagrams indicating various intermolecular proton-proton interactions in a dimer of a peptide analog in accordance with this invention.

FIG. 3 is a molecular diagram indicating various intramolecular proton-proton interactions in a peptide analog in accordance with this invention.

FIG. 4 is a plot showing the concentration dependence of NH chemical shifts for various NH groups in a peptide analog in accordance with this invention.

FIG. 5 is a plot showing CD (circular dichroism) spectra for several peptide analogs in accordance with this invention together with one peptide that is not included in this invention.

### DETAILED DESCRIPTION OF THE INVENTION AND PREFERRED EMBODIMENTS

Definitions

The term “β-strand conformation” is used herein to denote the three-dimensional conformation of a single peptide strand in which the strand is elongated such that its amide groups form a planar zig-zag backbone and the amino acid side chains extend out of the plane to either or both sides. A peptide strand may assume this conformation either on its own or in combination with another peptide (or peptide analog) in a β-sheet-like conformation as defined below.

The term “β-sheet-like interaction” is used herein to denote the interaction between two peptides both of which are in a β-strand conformation, in which the two strands are side-by-side in anti-parallel directions with hydrogen bonding between carbonyl groups in one backbone and amino groups in the other (and vice versa). The term also extends to the analogous interaction that occurs when one of the peptides is replaced by a peptide analog or another elongated molecule in which similar hydrogen bonds are formed along the lengths of the molecules. Any peptide analog in accordance with this invention may thus enter into a β-sheet-like interaction with a peptide, with itself, or with another peptide analog. An individual peptide may engage in β-sheet-like interactions with two such peptides, analogs or other molecules, one on each side of the first peptide.

The term “β-turn” is used herein to denote a sharp 180-degree (“hair-pin”) turn in a peptide sequence that places the segments on either side of the turn in sufficient proximity to engage in hydrogen bonding between opposing units in the segments such that the segments align to form a β-sheet-like interaction. In recitations of a linkage that “permits . . . a β-sheet-like interaction,” “permits a hair-pin turn,” and similar phrases, the word “permit” denotes that the linkage is capable of adopting a β-turn conformation with little or no resistance, as opposed to linkages that offer steric or electronic resistance to adopting a β-turn conformation.

The term “peptide” is used herein to denote a compound containing two or more of amino acid residues joined by an amide bond formed from the carboxyl group of one residue and the amino group of the adjacent residue. The term “amino acid” includes both naturally occurring and synthetic amino acids, as well as amino acid analogs and amino acid mimetics whose properties are similar to those of the naturally occurring amino acids. Naturally occurring amino acids are those that are encoded by the genetic code, as well as those that are modified after expression, such as hydroxyproline, carboxyglutamate, O-phosphoserine, and glycosylated amino acids. Amino acid analogs are compounds having functionalities similar to those of naturally occurring amino acids, i.e., an amino group, a carboxyl group, and one or more side chains attached to a framework of from 1 to 4 carbon atoms. Many such analogs are known to those skilled in the art, including but not limited to homoserine, norleucine, methionine sulfoximine, phenylglycine, (p-fluorophenyl)alanine, β-alanine, α-aminoisobutyric acid, tert-leucine, and β-methylaspartic acid.

The term “amino acid side chain” denotes the group represented by the “R” in the amino acid formula

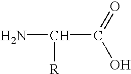

and includes any of the side chains in naturally occurring amino acids as well as those in modified amino acids and amino acid mimetics.

The term “activated leaving group” is used herein to denote a radical or group of atoms that is displaced from a carbon atom by the attack of a nucleophile in a nucleophilic substitution reaction.

The term “protecting group” is used herein to denote a radical or group of atoms that is bound to a particular atom of a molecule to prevent that atom from participating in reactions occurring on other portions of the molecule.

The term “amine protecting group” is used herein to denote a radical or group of atoms that is bound to an amine nitrogen atom of a molecule to prevent that nitrogen atom from participating in reactions occurring on other portions of the molecule. The term “amine-protected” denotes the structural characteristic of a molecule containing an amine nitrogen atom by which that nitrogen atom is prevented from participating in reactions occurring on other portions of the molecule.

The term “carboxy protecting group” is used herein to denote a radical or group of atoms that is bound to a carboxy oxygen atom of a molecule to prevent that oxygen atom from participating in subsequent reactions occurring on other portions of the molecule. The term “carboxy-protected” denotes the structural characteristic of a molecule containing a carboxy oxygen atom by which that oxygen atom is prevented from participating in reactions occurring on other portions of the molecule.

The term “solid support” is used herein to denote any inert solid that can be used to facilitate the separation of bound species from free species in a binding interaction such as a chromatographic separation or any of various analytical procedures that involve affinity-type binding. Solid supports include particles such as those used in chromatography columns as well as the inner walls of reaction vessels such as test tubes and the wells of microtiter plates, and other configurations well known to clinicians and laboratory technicians. Examples of materials used as solid supports are agarose, polystyrene, polyacrylamide, and these materials modified by poly(ethylene glycol). A peptide analog can be attached to these supports through the C-terminus (for example by an ester or amide linkage), through the N-terminus (for example, by a urea or carbamate linkage), or through a functionalized side chain (for example, by ester, amide, urea, carbamate, disulfide, or ether linkages).

Compounds, Peptide Analogs, and Constructs of The Invention

The 1,2-dihydro-3(6H)-pyridinone (azacyclohexenone) unit, which forms the nucleus of the present invention, has the molecular structure shown between the brackets in the following formula:

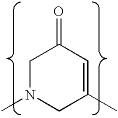

The functionalized azacyclohexenones of this invention are those having the formula

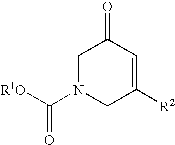

in which R¹ is a protecting group other than methyl or ethyl, and R² is either OH or an activated leaving group.

Peptide analogs of this invention include compounds of the following formulas

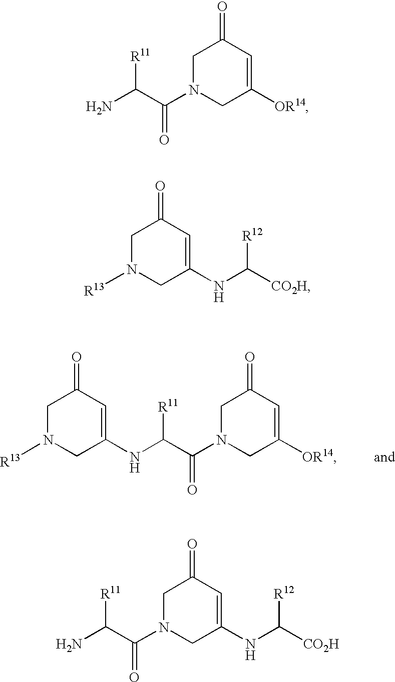

in which R¹¹ and R¹² are amino acid side chains, R¹³ is either H or an amine protecting group, and R¹⁴ is either H or a carboxy protecting group, and amine-protected analogs of the compounds that terminate in H₂N—, carboxy-protected analogs of the compounds that terminate in —CO₂H, carboxy-activated analogs of compounds that terminate in —CO₂H, amine-protected and carboxy-protected analogs of

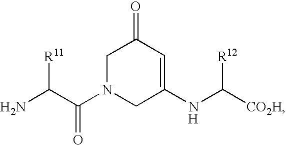

and amine-protected and carboxy-activated analogs of

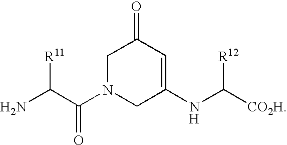

Among the above peptide analogs, one preferred subclass is that defined by the formula

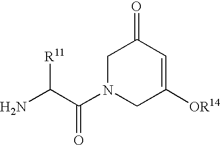

in which R¹⁴ is a carboxy protecting group, including amine-protected analogs of this formula. Another preferred subclass is that defined by the formula

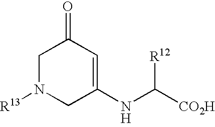

in which R¹³ is an amine protecting group, including carboxy-protected analogs of this formula. A further preferred subclass is that defined by the formula

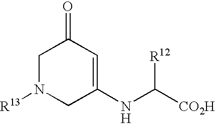

in which R¹³ is an amine protecting group, including carboxy-activated analogs of this formula. A still further preferred subclass is that defined by the formula

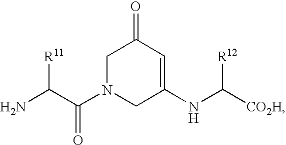

including amine-protected analogs, carboxy-protected analogs, carboxy-activated analogs, amine-protected and carboxy-protected analogs, and amine-protected and carboxy-activated analogs of this formula. The peptide analogs within these preferred classes are useful as components in the synthesis of longer-chain peptide analogs. In certain embodiments of this invention, the R¹¹ and R¹² groups in these formulas are side chains of natural amino acids. In other embodiments, either R¹¹, R¹², or both are unnatural amino acids.

Further peptide analogs of this invention are defined as peptides in which at least one amino acid, but less than all amino acids, is replaced by the azacyclohexenone group shown above. Preferred analogs are those containing from 2 to 200 amino acids and from 1 to 100 azacyclohexenone groups. More preferred are those analogs that contain from 2 to 100 amino acids and from 1 to 50 azacyclohexenone groups, and most preferred are those that contain from 2 to 10 amino acids and from 1 to 20 azacyclohexenone groups. The number ratio of azacyclohexenone groups to amino acids in these analogs is preferably from 1:10 to 10:1, more preferably from 1:5 to 5:1, and most preferably from 1:2 to 1:1.

Still further peptide analogs of this invention are defined by the following formula

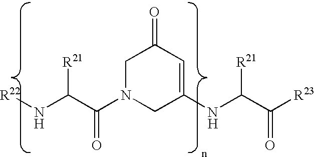

in which:

the R²¹'s are the same or different and each R²¹ is an amino acid side chain;

R²² is either a peptide chain terminating group or

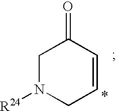

in which R²⁴ is either H, alkyl, acyl, carbamoyl, or alkoxycarbonyl, and * denotes the site of attachment;

R²³ is either a peptide chain terminating group or

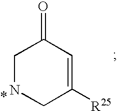

in which R²⁵ is either hydroxyl, alkoxy, alkylamino, dialkylamino, or arylamino, and * denotes the site of attachment; and

n is at least 2.

Preferred subclasses among these peptide analogs are those in which the R²¹'s are a combination of side chains of natural and unnatural amino acids and those in which the R²¹'s are all side chains of natural amino acids. Further preferred subclasses are those in which R²² is either acyl, carbamoyl, or alkoxycarbonyl. A preferred acyl group is acetyl. A still further preferred subclass is that in which R²² is

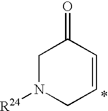

In terms of the R²³ group, a preferred subclass is that in which R²³ is either hydroxyl, alkoxyl, alkylamino, dialkylamino, or arylamino, with hydroxyl and methylamino most preferred. A still further preferred subclass is that in which R²³ is

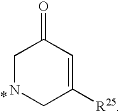

Among the peptide analogs containing the symbol “n” as an index of chain length, a preferred sub class is that in which n is 2 to 100, more preferred is that in which n is 2 to 50, and most preferred is that in which n is 2 to 5.

Constructs or hybrids in accordance with this invention include two sequences linked together by a linkage that permits a β-turn, the first sequence being a sequence of amino acids joined together by amide bonds as in a conventional peptide, and the second sequence being a sequence of amino acids joined together by amide bonds as in the first sequence except that one or more, but not all, of the amino acids is replaced by an azacyclohexenone unit. The azacyclohexenone unit(s), with the assistance of the covalent linkage, induces a β-sheet interaction between the two sequences and thereby induces and stabilizes the first, all-amino-acid, sequence in a β-strand conformation. In this configuration, the all-amino acid sequence is particularly effective in engaging in β-sheet interactions with other (“target”) peptides and thus performing such functions as inhibiting the target peptides from entering into β-sheet interactions with further peptides and thereby inhibiting the biological activity of these target peptides, and various affinity-type functions such as extracting the target peptides from peptide mixtures or mixtures in general. The construct size (i.e., the lengths of the two segments) is not critical to the invention, but in preferred embodiments, the all-amino-acid segment will contain from 3 to 200 amino acids and in the segment containing both amino acids and azacyclohexenone units the total of the acids and azacyclohexenone units will range from 3 to 200. Ranges for both segments that are more preferred are 3 to 100, and most preferred are 3 to 20. The linkage between the segments can vary and is not critical except that the linkage should not be one that is sterically or otherwise hindered from assuming a β-turn conformation. Preferred linkages are those that favorably assume or promote a β-turn conformation. Examples are D-proline-alanine (D-Pro-Ala) and asparagine-glycine (Asn-Gly).

In the constructs of this invention as well as the peptide analogs that are intended to enter into β-sheet-like interactions with target peptides, the amino acids of the azacyclohexenone-containing sequence are preferably those whose side chains are chosen on the basis of known side chain-side chain affinities within β-sheets through design of sterically and electronically complementary structures, or by screening analogs. See, for example, Smith, C. K., et al., “Guidelines for Protein Design: The Energetics of β-Sheet Side Chain Interactions,” Science 1995, vol. 270, 980; Ramirez-Alvarado,M., et al., “De novo design and structural analysis of a model β-hairpin peptide system,” Nature Structural Biology 1996, vol. 3, 604; von Heijne, G., et al., “The β-Structure: Inter-Strand Correlations,” J. Mol. Biol. 1997, vol. 117, 821. Thus, in accordance with known principles, the side chains of the amino acids in the azacyclohexenone-containing sequence preferably do not repel, but are instead compatible with, the side chains of the amino acids at the corresponding locations of the all-amino-acid segments or target peptides. This complementarity may result from a pairing of directly opposing residues but the affinity of any particular residue for an opposing residue may also be influenced by neighboring residues. Some of the ways in which directly opposing residues can be selected to achieve compatibility are the inclusion of basic side chains in the azacyclohexenone-containing sequence to oppose acidic side chains in the conventional peptide (all-amino-acid) sequence, acidic side chains in the azacyclohexenone-containing sequence to oppose basic side chains in the conventional peptide sequence, hydrophobic side chains in one sequence to oppose hydrophobic side chains in the other sequence, and hydrophilic side chains in the one sequence to oppose hydrophilic side chains in the other sequence. The characters of the side chains of known amino acids are well known to those skilled in the art and hence the appropriate selection for optimal favoring of β-sheet interaction will be readily apparent on this basis. The following is a rough characterization of several amino acids:

| Side Chain Character | Amino Acids |
|---|---|
| acidic | aspartic acid, glutamic acid |
| basic | arginine, histidine, lysine |
| hydrophobic | alanine, isoleucine, leucine, methionine, |
| | phenylalanine, valine, tryptophan, tyrosine |
| hydrophilic | asparagine, glutamine, serine, threonine |

Synthesis of the Compounds, Peptide Analogs, and Constructs of the Invention

The azacyclohexenones and their functionalized derivatives can be synthesized by conventional methods using 3,5-dimethoxypyridine, for example, as a starting material. In one such method, sodium borohydride is added to an acetonitrile solution of 3,5-dimethoxypyridine at −45° C., followed by addition of allyl chloroformate, to afford an intermediate N-acyl dihydropyridine which can be hydrolyzed directly to the protected enolic dione

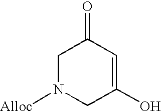

in which “Alloc” denotes the protecting group allyloxycarbonyl. The hydroxyl group is then activated by mesitylenesulfonyl chloride to form the mixed anhydride, and the activated compound is then coupled to an amino acid (ester) in tetrahydrofuran with the use of either ytterbium triflate or tin triflate as a catalyst. Coupling reactions of this type are described by Pérez, M., et al., Tetrahedron 1995, vol. 51, 8355; Laszlo, P., Tetrahedron Lett. 1989, vol. 30, 3969; and Matsubara, S., et al., Chem. Lett. 1994, 827. The resulting adduct can be N-deprotected and coupled to another amino acid using conventional procedures to form a tertiary amide. Alternatively, the ester can be deprotected and the resulting acid then coupled as a unit for more rapid chain elongation.

Coupling can also be performed by solid phase synthesis. For example, an Fmoc-protected amino acid coupled to a solid resin such as a Merrifield polystyrene can be deprotected with 20% pyridine in DMF, then coupled to an activated and N-protected form of the azacyclohexenone in the presence of tin triflate and DIEA in a mixed solvent of methylene chloride and DMF (1:3.5 volume ratio), followed by treatment with acetic anhydride, DIEA, and methylene chloride (1:1:3). The N-protecting group is then removed, and the steps repeated until the desired peptide analog chain is achieved.

The level of formation of N-allylated peptide analogs, which are impurities in the product, will vary with the choice of scavenging reagent for the palladium-catalyzed Alloc deprotection of the resin-bound peptide analogs. When N-methylmorpholine (NMM) in acetic acid-chloroform (37:1:2 CHCl₃:NMM:AcOH) is used as the scavenging reagent, a significant quantity of the N-allylated impurities may be formed. However, when Me₃SiN(Me)₂ is used as the scavenging reagent, the formation of these impurities is suppressed.

Constructs consisting of an all-amino-acid segment linked to a segment in which one or more (but not all) amino acids are replaced by an azacyclohexenone group are readily synthesized by methods analogous to those described above, with the β-turn-promoting linkage added at the appropriate site. Solid phase synthesis is readily used, and the azacyclohexenone units can be incorporated at either the N-termini or the C-termini of the hybrid. C-terminal azacyclohexenone incorporation, for example, can be performed by solid-phase synthesis of the desired azacyclohexenone-containing segment, followed by incorporation of the amino acids using standard peptide coupling conditions. N-terminal azacyclohexenone incorporation can be performed by first synthesizing the solid-phase-bound peptide segment, followed by incorporation of the azacyclohexenone-containing segment. The synthesis of larger constructs , such as those incorporating two or more azacyclohexenone units separated by one or more amino acids, is best achieved by preassembling the segments, preferably in dimeric form, and then linking them together, since as the construct grows in length it tends to assume a β-sheet conformation of its own, thereby inhibiting coupling efficiency.

Formulations and Administration of the Peptide Analogs and Constructs of the Invention

When used as drugs for administration to mammals, the compounds of this invention can be administered in water-soluble form, in which case they are often used in the form of pharmaceutically acceptable salts. Pharmaceutically acceptable salts are those that retain the biological effectiveness of the free bases or acids without introducing unfavorable side effects. The salts can be either acid or base addition salts, depending on the peptide analog itself. Examples of acceptable acid addition salts are those formed with inorganic acids such as hydrochloric, hydrobromic, sulfuric, nitric, or phosphoric acid, and those formed with organic acids such as acetic, propionic, glycolic, pyruvic, oxalic, maleic, malonic, succinic, fumaric, tartaric, citric, benzoic, cinnamic, mandelic, methanesulfonic, ethanesulfonic, p-toluenesulfonic, or salicylic acid. Examples of acceptable base addition salts are those formed with inorganic bases such as sodium, potassium, lithium, ammonium, calcium, magnesium, iron, zinc, manganese, or aluminum hydroxide, and those formed with organic bases such as primary, secondary, and tertiary amines such as isopropylamine, trimethylamine, diethylamine, triethylamine, tripropylamine, and ethanolamine, or with basic ion exchange resins.

The compounds can be formulated into suitable pharmaceutical preparations for administration by intravenous injection, intramuscular injection, intravenous infusion, oral administration, or any other conventional methods of administration. The active ingredient can be compounded, for example, with the usual non-toxic, pharmaceutically acceptable carriers and excipients as aqueous solutions, or as emulsions or suspensions, or in solid or semi-solid forms such as tablets, pellets, capsules, or suppositories. Typical carriers are water, glucose, lactose, gum acacia, gelatin, mannitol, starch paste, magnesium trisilicate, talc, corn starch, keratin, colloidal silica, potato starch, urea. Excipients can include agents for stabilization, thickening, coloring, or scent, or agents to aid in formulating the dosage forms, selected as needed in accordance with the intended manner of administration as well as the particular condition to be treated. Tablets for oral administration, for example, can contain microcrystalline cellulose, sodium citrate, calcium carbonate, dicalcium phosphate, or glycine, along with any of various disintegrants such as corn, potato, or tapioca starch, alginic acid or complex silicates, together with granulation binders such as polyvinylpyrrolidone, sucrose, gelatin or acacia. Lubricating agents such as magnesium stearate, sodium lauryl sulfate or talc can also be included.

The amount of active ingredient to be included in a single dosage form will vary depending on the patient to be treated and the particular mode of administration. The optimal dose level for a particular patient will depend on such factors as the age, body weight, general health, sex, and diet of the patient, as well as the time of administration, the route of administration, the rate of excretion, the severity of the disease being treated, and whether or not the patient is simultaneously undergoing any other drug therapy. In most cases, the amount of active ingredient administered will range from about 1 to about 1,000 mg per day, preferably from about 10 to about 500 mg per day.

The following examples are offered for purposes of illustration, and are not intended to impose limits on the scope of the invention.

The reagents used in these examples were obtained from commercial suppliers and used as received. Solvents that were not obtained from commercial suppliers in anhydrous form were dried by distillation prior to use. Flash chromatography was performed using 60-mesh silica gel. In the following descriptions, the abbreviation “Ach” denotes the 1,2-dihydro-3(6H) pyridinyl (azacyclohexenone) unit. Other abbreviations used are as follows:

| | |
|---|---|
| Ac₂O | acetic anhydride |
| AcOH | acetic acid |
| CD | circular dichroism |
| COSY | correlated spectroscopy |
| DEPT | distortionless enhancement by polarization transfer |
| DIEA | diisopropylethylamine |
| DMAP | dimethylaminopyridine |
| EDC | ethyl 3-(dimethylamino)propyl carbodiimide |
| Et | ethyl |
| EtOAc | ethyl acetate |
| HMQC | heteronuclear multiple-quantum coherence |
| Me | methyl |
| Mes | mesitylene |
| NMM | N-methyl morpholine |
| NOE | nuclear Overhauser effect |
| NOESY | nuclear Overhauser effect spectroscopy |
| PyBroP | bromotris(pyrrolidino)phosphonium |
| | hexafluorophosphate |
| TFA | trifluoroacetic acid |
| THF | tetrahydrofuran |
| TOCSY | total correlation spectroscopy |

NMR spectra were obtained using a Bruker 500 MHz spectrometer in CDCl₃ solution unless otherwise indicated. Spectral data are reported as chemical shifts (multiplicity, number of hydrogens, coupling constants in Hz). ¹H NMR chemical shifts are referenced to TMS (0 ppm) in CDCl₃, CD₃OD (3.31 ppm), or (CD₃)₂CO (2.05 ppm); ¹³C NMR spectra were proton decoupled and referenced to CDCl₃ (77.16 ppm), or CD₃OD (49.00 ppm). Resonance assignments were obtained by the method of Wüthrich, K., NMR of Proteins and Nucleic Acids; John Wiley & Sons: New York, 1986, using TOCSY and NOESY spectra. Samples were analyzed at approximately 20 mM in CD₃OH/CDCl₃ solutions. Rigorous degassing was performed prior to the NOESY experiments using the freeze-pump-thaw method. NOESY experiments were performed with mixing times optimized to limit spin-diffusion (0.7 s). NOESY data were collected with 2048 data points in F2 and 512 data points in F1.

### EXAMPLE 1

This example illustrates the liquid-phase synthesis of an acyl- and methylamine-terminated peptide analog in accordance with this invention containing three amino acids and two Ach units in alternating positions along the peptide chain.

### A. Synthesis of Prop-2-enyl 5-Hydroxy-3-oxo-1,2,6-trihydropyridine-1-carboxylate (an N-Protected Ach Unit)

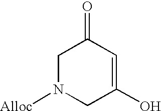

To a solution of 3,5-dimethoxypyridine (8.5 g, 61 mmol) in dry MeCN (230 mL) at −45° C. was added NaBH₄ (4.16 g, 110 mmol) in portions over 10 min, and the resulting mixture was stirred for an additional 10 min. Allyl chloroformate (7.79 mL, 73.4 mmol) was added over 45 min while the temperature (measured by an internal thermometer) was maintained within the range of −45 to −40° C. The reaction was allowed to proceed for an additional 15 min at −40° C., and then 1 N HCl (150 mL) was added at −40° C. The HCl addition was followed immediately by addition of saturated NaHCO₃ (100 mL) until the pH was basic. The aqueous layer was extracted with EtOAc (3×50 mL), and the organic layer was dried over Na₂SO₄ and evaporated in vacuo. The crude product was dissolved in THF (200 mL) and 1 N HCl (200 mL). The reaction mixture was stirred for 30 min at room temperature and then made basic with solid NaOH at 0° C. The aqueous layer was washed once with EtOAc (50 mL), and the organic layer was subsequently washed with 1 N NaOH until the aqueous layer was no longer yellow. The combined aqueous layers were acidified with 6N HCl at 0° C., saturated with NaCl, and extracted three times with EtOAc (50 mL). The combined organic layer was dried over Na₂SO₄ and concentrated to a thick oil. The enolic diketone tended to decompose on standing, so the crude product (9.5 g, 48 mmol, ca. 79%) was used immediately in the following step. An analytical sample was purified by flash chromatography using a gradient of petroleum ether/EtOAc to give the enolic diketone prop-2-enyl 5-hydroxy-3-oxo-1,2,6-trihydropyridine-1-carboxylate as an oil. Confirmation of the structure as that shown above was achieved as follows: ¹H NMR δ 4.20 (s, 4), 4.64 (d, 2, J=5.3), 5.27 (d, 1, J=19), 5.31 (d, 1, J=25), 5.63 (s, 1), 5.87-6.00 (m, 1), 9.90 (br s, 1); ¹³C NMR δ 46.56, 47.44, 66.89, 102.84, 118.31, 131.95, 154.73, 184.93, 186.86; HRMS (FAB) m/z 198.0767 (M+H⁺, C₉H₁₁NO₄ requires 198.0766).

### B. Activation of Prop-2-enyl 5-Hydroxy-3-oxo-1,2,6-trihydropyridine-1-carboxylate at the 5-Position by Forming Prop-2-enyl 3-Oxo-5-[(2,4,6-trimethylphenyl)sulfonyloxy]-1,2,6-trihydropyridine-1-carboxylate

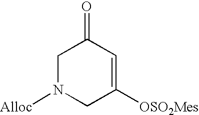

To a stirring solution of enolic diketone whose preparation is described in the preceding paragraph (9.5 g, 48 mmol), in anhydrous CH₂Cl₂ (150 mL) under a nitrogen atmosphere, was added powdered anhydrous K₂CO₃ (10.97 g, 79.50 mmol) and mesitylenesulfonyl chloride (15.8 g, 72.3 mmol). After 4 h, excess reagent was quenched by addition of saturated NH₄Cl (100 mL). The aqueous phase was washed three times with CH₂Cl₂ (100 mL), and the combined organic phases were washed with brine, dried over (Na₂SO₄), and concentrated under vacuum. The crude product was chromatographed (EtOAc/hexanes 1:2) to yield the mixed anhydride prop-2-enyl 3-oxo-5-[(2,4,6-trimethylphenyl)sulfonyloxy]-1,2,6-trihydropyridine-1-carboxylate (9.1 g, 24 mmol, 69%) as a pale yellow oil. The product was found to be stable at room temperature in a 0.1 M CH₂Cl₂ solution, but for prolonged storage the compound was dissolved in CH₂Cl₂ (1 M) and kept at −78° C. Confirmation of the structure as that shown above was achieved by the following: ¹H NMR δ 2.35 (s, 3), 2.63 (s, 6), 4.09 (s, 2), 4.32 (s, 2), 4.62 (d, 2, J=5.5), 5.24 (d,1, J=10.6), 5.30 (d, 1, J=17.5), 5.83-5.98 (m, 1), 7.05 (s, 2); ¹³C NMR δ 20.92, 22.49, 44.02, 50.30, 66.69, 113.78, 118.12, 129.93, 131.86, 132.03, 139.94, 144.94, 154.15, 191.96; MS (FAB) m/z (%)=144 (100), 323 (70), 380 (30, M+H⁺).

### C. Coupling of the Activated Compound to Isoleucine tert-Butyl Ester to Form tert-Butyl (2S,3S)-3-Methyl-2-{[5-oxo-1-(prop-2-enyloxycarbonyl)-1,2,6-trihydro-3-pyridyl]amino}pentanoate (Alloc-Ach-Ile t-Butyl Ester)

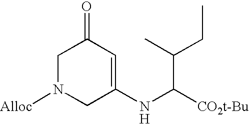

To a solution of anhydride of the preceding paragraph (1.0 g, 2.6 mmol) in dry THF (11 mL) were added isoleucine tert-butyl ester hydrochloride (0.5 g, 2.7 mmol), anhydrous ytterbium(III) triflate (1.64 g, 2.65 mmol), and DIEA (1.38 mL, 7.92 mmol) under a nitrogen atmosphere. After 24 h, saturated NH₄Cl was added (10 mL) and the mixture was extracted with EtOAc (3×10 mL). The combined organic extracts were washed with brine, dried over MgSO₄, and evaporated. Purification of the crude product by flash chromatography (hexanes/EtOAc 1:1) gave the vinylogous amide shown above (0.70 g, 1.9 mmol, 73%) as a light yellow oil. Proton and carbon spectra showed peak doubling due to amide bond rotamers while confirming the structure of the compound: ¹H NMR δ 0.89-0.98 (m, 6), 1.47-1.49 (s, 9, rot), 1.49-1.63 (m, 1); 1.65-1.78 (m, 1); 1.83-1.93 (m, 1); 3.88 (dd, 1, J=4.9, J=7.7), 4.02 (d, 1, J=17.9), 4.10 (d, 1), 4.27 (d, 1, J=16.1), 4.38 (d, 1, J=16.6); 4.63 (d, 2, J=5.5), 5.18 (s, 1), 5.23 (d, 1, J=10.4), 5.31 (m, 1, J=17.3, J=1.6, J=3.1), 5.84 (d, 1, J=6.9), 5.83-5.99 (m, 1); ¹³C NMR δ 11.55, 11.67, 14.06, 14.83, 15.53, 24.69, 25.96, 27.94, 27.97, 37.31, 39.18, 44.22, 50.57, 59.28, 59.46, 66.53, 80.67, 82.95, 95.64, 117.83, 174.71; MS (FAB) m/z (%)=450 (100), 367 (M⁺+H), 338 (42), 311 (84), 292 (22), 265 (20), 244 (28), 225 (20), 198 (32), 179 (10), 154 (18); HRMS (FAB) m/z 367.2232 (MH⁺, C₁₉H₃₀N₂O₅ requires 367.2233).

### D. Deprotection of the Coupling Product to Form tert-Butyl (2S,3S)-3-Methyl-2-[(5-oxo-1,2,6-trihydro-3-pyridyl)amino]pentanoate (Ach-Ile t-Butyl Ester)

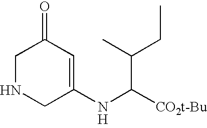

To a solution of the Alloc-amine of the preceding paragraph (0.46 g, 1.3 mmol) in a 1:1 mixture of THF/diethylamine (4.8 mL) at room temperature was added tetrakis(triphenyl-phosphine)palladium(0) (0.12 g, 0.11 mmol). The resulting mixture was stirred for 1 h. The solvent was then evaporated, 1N HCl (25 mL) was added, and the new solution was washed three times with EtOAc. The aqueous phase was brought to pH>14 with solid NaOH and extracted with three portions of EtOAc. The pH was readjusted to pH>14 and the extraction was repeated. The combined organic extracts were washed with brine and with brine containing diethyl dithiocarbamic acid, dried over Na₂SO₄, and evaporated to afford the crude amine, which was used immediately in the next step. An analytical sample was purified by flash chromatography using CH₂Cl₂/MeOH (9:1) containing 3% Et₃N. Conformation of the structure of the product as that shown above was achieved by the following: ¹H NMR δ 0.91 (d, 3, J=6.6), 0.96 (t, 3, J=7.5), 1.31-1.38 (m, 1H), 1.49 (s, 9), 1.51-1.61 (m, 1), 1.81-1.91 (m, 1), 3.39 (s, 2), 3.60 (s, 2), 3.86 (dd, 1, J=4.6, J=7.5), 5.11 (s, 1), 5.59 (d, 1, J=7.7); ¹³C NMR δ 11.54, 14.84, 25.94, 27.94, 37.28, 47.20, 53.23, 59.20, 82.93, 95.39, 162.73, 170.33, 195.98; MS (FAB) m/z (%)=338 (48), 292 (30), 283 (M+H⁺, 74), 227 (66); the mass spectrum also showed aggregates with masses higher than M⁺.

### E. Coupling of Ach-Phe tert-Butyl Ester to Fmoc-Isoleucine to Form Fmoc-Ile-Ach-Phe, t-Butyl Ester

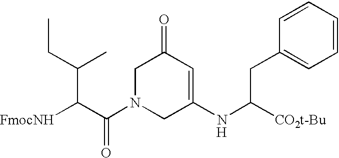

In a procedure analogous to that described above for tert-butyl (2S,3S)-3-methyl-2-{[5-oxo-1-(prop-2-enyloxycarbonyl)-1,2,6-trihydro-3-pyridyl]amino}pentanoate (Alloc-Ach-Ile t-butyl ester), the Phe analog, tert-butyl (2S, 3S)-3-phenyl-2-{[5-oxo-1-(prop-2-enyloxycarbonyl)-1,2,6-trihydro-3-pyridyl]amino}propanoate (Alloc-Ach-Phe t-butyl ester), was prepared. Once this compound was prepared, it was deprotected by treating a solution of the compound (100 mg, 0.25 mmol) in a 1:1 mixture of THF/diethylamine (2 mL) at room temperature with tetrakis(triphenylphosphine)palladium(0) (28 mg, 0.03 mmol). The resulting mixture was stirred for 1 h, following which the solvent was evaporated under reduced pressure, then co-evaporated under reduced pressure from dioxane (2×2 mL) to afford the crude amine (the deprotected Ach-Phe t-butyl ester). To this amine (80 mg, 0.25 mmol) in CH₂Cl₂ (3.5 mL) was immediately added Fmoc-isoleucine (0.18 g, 0.51 mmol) and DIEA (0.44 mL, 2.5 mmol). The reaction mixture was stirred at room temperature under a nitrogen atmosphere for 26 h, then evaporated under reduced pressure. The residue was redissolved in EtOAc and the solution was washed with 1 M HCl (3×3 mL), NaHCO₃ (1×3 mL), and brine (1×3 mL), dried over MgSO₄ and concentrated. The crude product was purified by flash chromatography (EtOAc:hexanes(2:1) to afford tri-@-tide (0.13 g, 0.21 mmol, 82%) as a light yellow oil. Confirmation of the structure as that of Fmoc-Ile-Ach-Phe, t-butyl ester was achieved by the following: ¹H NMR δ 0.59 (bs, 0.3), 0.68 (bs, 0.3), 0.88 (m, 6), 1.15 (m, 1), 1.37 (s, 9), 1.50 (bm, 1), 1.60 (bm, 1), 3.13 (m, 2), 4.06 (m, 2), 4.18 (m, 2), 4.32 (m, 3), 4.56 (m, 1), 5.06 (s, 0.5), 5.09 (s, 0.5), 5.23 (s, 0.2), 5.28 (s, 1), 5.62 (d, 0.2), 5.71 (d, 0.2), 6.23 (d, 1), 6.54 (bs, 1), 7.12 (m, 2), 7.24 (m, 6), 7.36 (m, 1), 7.46 (m, 1), 7.55 (m, 1), 7.67 (m, 1), 7.74 (d, 1); ¹³C NMR δ 11.19, 15.67, 24.25, 37.33, 37.60, 42.86, 47.11, 52.66, 54.87, 56.43, 83.64, 95.20, 119.92, 125.08, 125.14, 126.89, 127.08, 127.30, 127.63, 128.42, 128.47, 128.52, 129.40, 131.90, 131.92, 132.02, 132.10, 132.88, 135.10, 141.22, 143.76, 143.91, 156.44, 159.96, 169.76, 171.74, 189.53; MS (FAB) m/z (%)=652 (55)(M+H⁺), 596 (20), 400 (10); HRMS (FAB) m/z 652.3394 (M+H⁺, C₃₉H₄₆N₃O₆ requires 652.3387).

### F. Synthesis of (2S)-4-Methyl-2-[5-oxo-1-(prop-2-enyloxycarbonyl)(3-oxo-1,2,6-trihydro-3-pyridyl)]amino]pentanoic acid (Alloc-Ach-Leu)

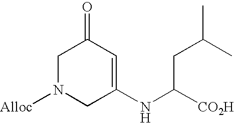

In a procedure analogous to that described above for tert-butyl (2S,3S)-3-methyl-2-{[5-oxo-1-(prop-2-enyloxycarbonyl)-1,2,6-trihydro-3-pyridyl]amino}pentanoate (Alloc-Ach-Ile t-butyl ester), the Leu analog, tert-butyl (2S,3S)-4-methyl-2-{[5-oxo-1-(prop-2-enyloxycarbonyl)-1,2,6-trihydropyridyl]amino}pentanoate (Alloc-Ach-Leu t-butyl ester) was prepared. Once prepared, this compound (2.75 g, 7.48 mmol) was dissolved in neat TFA (25 mL) under argon and stirred for 2 h. After evaporation of the solvent, EtOAc was added and the solution was washed with two portions of saturated NaH₂PO₄ and brine, dried over Na₂SO₄, and evaporated. The residue was purified by flash chromatography using a gradient of petroleum ether/EtOAc/AcOH (79:20:1, then 0:99:1); traces of acetic acid were removed by co-evaporation with three portions of toluene to give the pure acid (2S)-4-methyl-2-{[5-oxo-1-(prop-2-enyloxycarbonyl)(3-oxo-1,2,6-trihydropyridyl)]amino}pentanoic acid (Alloc-Ach-Leu) as a yellow oil in quantitative yield (2.32 g, 7.48 mmol). The structure was confirmed by the following: ¹H NMR δ 0.92 (d, 3, J=5.0), 0.96 (d, 3, J=5.2), 1.68-1.79 (m, 3), 4.01-4.20 (m, 2), 4.12 (dd, 1, J=7.2, J=7.2), 4.34 (d, 1, J=17.4), 4.40 (d, 1, J=16.8), 4.61 (s, 2), 5.23 (d, 1, J=10.5), 5.30 (d, 1, J=16.9), 5.37 (s, 1), 5.86-5.94 (m, 1), 6.80 (bs, 1); ¹³C NMR δ 21.74, 22.57, 24.86, 40.46, 44.01, 54.52, 60.49, 67.01, 94.42, 118.35, 131.91, 154.88, 164.02, 171.40, 174.21; MS (FAB) m/z (%)=311 (M⁺, 100), 265 (20), 225 (33), 154 (86), 136 (74), 107 (34); HRMS (FAB) m/z 311.1615 (M+H⁺, C₁₅H₂₂N₂O₅ requires 311.1607).

### G. Coupling of Alloc-Ach-Leu to Ach-Ile, tert-Butyl Ester

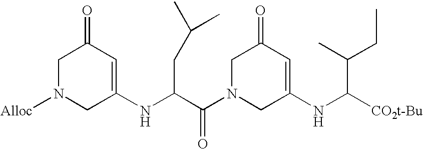

A solution was prepared, containing Alloc-Ach-Leu (1.66 g, 5.35 mmol) and Ach-Ile t-butyl ester(1.51 g, 5.35 mmol), whose preparations are described in the paragraphs above, in dry CH₂Cl₂ (40 mL). While maintaining the solution at 0° C. by an ice bath, the solution was treated by the addition of the reagents DIEA (1.67 mL, 9.63 mmol), 4-DMAP (63 mg, 535 μmol), and PyBroP (3.24 g, 6.96 mmol). After 30 minutes, the ice bath was removed, and the mixture was stirred for 14 h at room temperature. After dilution with CH₂Cl₂, the solution was extracted with four portions of 1N HCl, saturated NaHCO₃, and brine, dried (MgSO₄), and evaporated. The crude product was purified by flash chromatography using a gradient of CH₂Cl₂/MeOH (97:3, 95:5) to give a fraction of pure Alloc-Ach-Leu-Ach-Ile, tert-butyl ester (1.28 g, 2.33 mmol, 42%) and a fraction (2.65 g) contaminated with tris(pyrrolidino)phosphoramide. Although the NMR spectra were complicated by peak doubling due to amide rotamers, the structure of the product was confirmed as that shown above, i.e., Alloc-Ach-Leu-Ach-Ile, tert-butyl ester, by the following: ¹H NMR δ 0.86-0.98 (m, 12), 1.45-1.49 (s, 9), 1.64-2.08 (m, 4), 3.88 (dd, 1, J=4.9, J=7.6), 4.00-4.38 (m, 6), 4.44 (dd, 1, J=4.9, J=8.2), 4.46 (d, 1, J=16.9), 4.57 (d, 1, J=16.9), 4.63 (d, 2, J=5.2), 5.20 (s, 1), 5.22 (ddd, 1, J=1.4, J=2.5, J=10.5), 5.30 (ddd, 1, 1.5, J=3.1, J=17.2), 5.39 (s, 1), 5.91 (ddt, 1, J=10.5, J=17.2, J=5.5), 6.08-6.17 (d, 1, J =7.8), 6.43 (s, 1); ¹³C NMR δ 12.31, 12.35, 15.67, 16.09, 23.45, 25.40, 25.93, 26.73, 28.68, 28.73, 38.12, 38.54, 42.34, 44.82, 52.40, 55.83, 57.73, 60.55, 61.07, 67.38, 82.65, 83.97, 95.80, 96.23, 118.52, 158.28, 170.39, 188.52; MS (FAB) m/z (%)=no M⁺, 480 (78), 424 (100), 323 (22), 265 (30), 225 (14), 179 (19); HRMS (FAB) m/z no M⁺ observed in FAB.

### H. Coupling of Alloc-Ach-Leu-Ach-Ile, tert-butyl ester, to Fmoc-Phenylalanine to Form Fmoc-Phe-Ach-Leu-Ach-Ile, tert-Butyl Ester

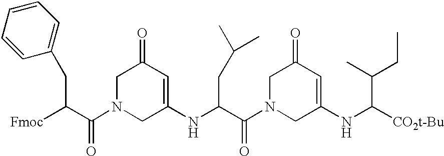

To a solution of tetramer of the preceding paragraph (1.10 g, 1.91 mmol, contaminated with tris(pyrrolidino)phosphoramide) in a 1:1-mixture of dry THF/Et₂NH (20 mL) was added tetrakis(triphenylphosphine)palladium(0) (20 μmol, 23 mg) under argon. After 4 h, 1N HCl was added to pH<1, and the mixture was extracted with three portions of EtOAc. The aqueous layer was brought to pH>14 with 5N NaOH and extracted with four portions of EtOAc. Washing with brine (containing ca. 200 mg of sodium diethyldithiocarbamate), drying over Na₂SO₄, and evaporation of the solvent gave a crude product, which was purified by flash chromatography (gradient of CH₂Cl₂-MeOH-Et₃NH 90:10:0, 80:20:3) to give the N-deprotected Ach-Leu-Ach-Ile tert-butyl ester (721 mg, 1.74 mmol, 77%) as a yellow solid, whose structure was confirmed by the following. ¹H NMR 67 0.87 (d, 3, J=5.6), 0.91 (d, 3, J=2.8) 0.92 (d, 3, J=3.2), 0.95 (t, 3, J=7.6), 1.30-1.34 (m, 1), 1.50 (s, 9), 1.52-1.69 (3), 1.71-1.79 (m 1), 1.84-1.92 (m, 1), 3.35 (s, 2), 3.58 (d, 1, J=16.9), 3.64 (d, 1, J=16.6), 3.91 (dd, 1, J=5.0, J=7.8), 4.00 (d, 1, J=17.1), 4.10 (d, 1, J=16.9), 4.26 (d, 1, J=17.1), 4.46-4.52 (m, 1), 5.14-5.16 (s, 1), 5.19-5.22 (s, 1), 7.02 (d, 1, J=8.1), 7.18 (d, 1, J=7.3); ¹³C NMR δ 12.23, 15.70, 22.05, 23.84, 25.37, 26.67, 28.65, 38.40, 41.63, 43.54, 46.56, 47.81, 51.17, 52.83, 54.15, 60.61, 83.68, 94.93, 95.11, 162.04, 165.38, 170.54, 171.78, 189.71, 196.36; MS (FAB) m/z (%)=491 (M+, 100), 435 (44), 340 (6), 319 (10), 281 (8), 225 (22), 179 (30), 154 (18), 136 (14), 111 (20); HRMS (FAB) m/z491.3233 (M⁺+H, C₂₆H₄₂N₄O₅ requires 491.3233).

To a solution of the N-deprotected compound resulting from the procedure of the last paragraph (510 mg, 1.04 mmol) in dry CH₂Cl₂ (5 mL) were added Fmoc-phenylalanine (603 mg, 1.56 mmol), PyBroP (726 mg, 1.56 mmol), 4-DMAP (6 mg, 52 μmol), and DIEA (723 μl, 4.16 mmol). The reaction mixture was stirred under argon at room temperature for 16 h, EtOAc was added, and the solution was washed with 1N HCl, saturated NH₄Cl and brine, dried over Na₂SO₄, and evaporated. Purification by flash chromatography (EtOAc/MeOH 95:5) gave the pentamer Fmoc-Phe-Ach-Leu-Ach-Ile, tert-butyl ester (812 mg, 944 μmol, 91%) as a white solid, whose structure was confirmed by the following: ¹H NMR δ 0.80-0.92 (m, 12), 1.23-1.32 (m, 1), 1.40 (s, 9), 1.44-1.63 (m, 4), 1.77-1.86 (m, 1), 2.85-2.93 (m, 2), 2.99 (s, 2), 3.69-4.48 (m, 10), 4.74-5.18 (m, 4), 6.96-7.24 (m, 7), 7.25-7.35 (m, 2), 7.38-7.51 (m, 2), 7.60-7.73 (m, 2); ¹³C NMR δ 11.50, 13.94, 14.91, 20.82, 22.10, 23.06, 24.57, 26.11, 27.81, 27.86, 37.93, 38.83, 41.14, 42.06, 46.86, 51.68, 51.85, 59.64, 60.41, 67.02, 83.58, 93.70, 94.16, 119.80, 124.91, 124.99, 126.77, 127.09, 127.55, 128.53, 129.00, 135.60, 141.16, 143.53, 143.68, 156.15, 160.95, 161.17, 169.93, 170.50, 171.31, 171.47, 188.97, 189.65; MS (FAB) m/z (%)=861 (M⁺, 48), 179 (100), 154 (84), 137 (58); HRMS (FAB) m/z 860.4611 (M⁺+H, C₅₀H₆₁N₅O₈ requires 860.4598).

### I. Conversion of Fmoc-Phe-Ach-Leu-Ach-Ile, tert-Butyl Ester, to Ac-Phe-Ach-Leu-Ach-Ile, N-Methyl Amide

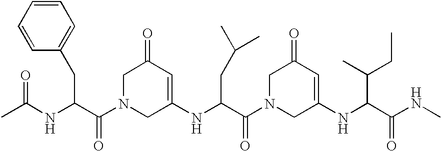

A solution of the Fmoc-pentamer of the preceding paragraph (749 mg, 871 μmol) in dry CH₂Cl₂ (5 mL) was treated with Et₂NH (5 mL) at room temperature under argon for 3 h. The solution was evaporated under reduced pressure, the residue was co-evaporated with three portions of dichloroethane (5 mL) and dried under high vacuum. The crude amine was redissolved in dry CH₂Cl₂ (5 mL), and dry pyridine (1.41 mL, 17.5 mmol) and acetic anhydride (831 μL, 8.71 mmol) were added. After 50 min, the volatile materials were removed under vacuum and the residue was co-evaporated with three portions of C₂H₄Cl₂ (5 mL). Purification of the crude product by flash chromatography (gradient of CH₂Cl₂-MeOH 95:5-9:1) as eluent gave the acetyl derivative (505 mg, 743 μmol, 85%) as a yellowish solid. The structure of the acetyl derivative was confirmed by the following: ¹H NMR (300 MHz, CDCl₃) δ 0.85-1.02 (m, 12), 1.28-1.43 (m, 1), 1.49-1.53 (s, 9), 1.64-1.74 (m, 1), 1.84-1.95 (m, 1), 2.01-2.06 (s, 3), 2.93 (d, 2, J=6.6), 3.80-4.72 (m, 10), 5.09-5.47 (m, 3), 6.94-7.29 (m, 5); ¹³C NMR (75 MHz, CDCl₃) δ 11.38, 14.96, 20.80, 22.02, 22.56, 23.11, 24.43, 25.92, 27.81, 38.02, 39.01, 41.95, 42.49, 42.69, 49.68, 50.37, 51.58, 51.78, 59.63, 82.50, 83.13, 93.64, 94.04, 126.89, 128.29, 128.89, 135.38, 161.06, 161.26, 169.91, 170.12, 170.47, 170.90, 188.91, 189.59; MS (FAB) m/z (%)=680 (M⁺, 100), 624 (30), 435 (30), 225 (36), 179 (54), 120 (62); HRMS (FAB) m/z 680.4012 (M⁺+H, C₃₇H₅₃N₅O₇ requires 680.4023).

The acetyl derivative (388 mg, 571 μmol) was dissolved in dichloroethane (3.5 mL) and treated with TFA (1.5 mL) for 5 h. The volatile materials were evaporated under reduced pressure, and the residue was co-evaporated with three portions of dichloroethane (5 mL) and dissolved in CH₂Cl₂. The solution was washed with saturated NaH₂PO₄, dried over Na₂SO₄, and evaporated to yield the crude acid (347 mg, 556 μmol, 97%) as a yellowish foam.

A solution of the crude acid and 1-hydroxy-7-azabenzotriazole (108 mg, 799 μmol), EDC (137 mg, 714 μmol), 4-DMAP (3.5 mg, 29 μmol), and methylamine (2.0 M in THF, 570 μL, 1.14 mmol) in dry CH₂Cl₂ (5 mL) was stirred under argon at 0° C. for 20 h at room temperature. CH₂Cl₂ was added, the solution was washed twice with 10% KHSO₄ and saturated NaHCO₃, and with brine, dried over Na₂SO₄, and evaporated to give 240 mg of crude methylamide. Purification by flash chromatography (gradient of CH₂Cl₂-MeOH 9:1-8:2) gave 154 mg (242 μmol, 42% over 2 steps) of Ac-Phe-Ach-Leu-Ach-Ile N-methylamide as a colorless solid: m.p. 210-215° C. (dec.). The structure of this product was confirmed by the following: ¹H NMR (CDCl₃-CD₃OH 10:1) δ 0.80-0.86 (m, 6), 0.87-0.94 (m, 6), 1.06-1.16 (m, 1), 1.54 (bs, 2), 1.57-1.64 (m, 1), 1.75-1.87 (m, 1), 1.89 (d, 0.5, J=6.4, rot), 1.95 (s, 3, rot), 2.71 (s, 3), 2.89 (dd, 1, J=8.9, J=12.5); 2.96 (dd, 1, J=6.9, J=13.3), 3.71 (d, 1, J=16.8), 3.82 (d, 1, J=16.8), 3.82-3.86 (m 1), 3.98-4.07 (m, 1), 4.07-4.11 (m, 2), 4.27-4.40 (m, 1), 4.53 (dd, 1, J=7.6, J=12.2); 4.82 (d, 1, J=17.6), 4.83 (d, 1, J=17.6), 5.01-5.09 (m, 0.3, rot), 5.21-5.25 (m, 1, rot), 5.19 (s, 1), 5.37 (s, 1), 7.08 (d, 2, J=6.4), 7.11-7.18 (m, 3), 7.35 (d, 1, J=9.0), 7.91 (d, 1, J=8.4), 8.05 (d, 1, J=4.4), 8.35 (bs, 1); ¹³C NMR δ 10.89, 14.93, 22.19, 22.47, 23.18, 24.34, 25.01, 25.78, 37.89, 39.19, 42.08, 42.45, 42.63, 49.58, 50.32, 51.29, 51.60, 60.40, 93.66, 126.79, 128.31, 128.86, 135.76, 160.60, 161.61, 170.79, 170.82, 170.93, 171.00, 189.13, 189.38; MS (FAB) m/z (%)=637 (M⁺, 100), 179 (64), 154 (32), 137 (28), 120 (68); HRMS (FAB) 637.3723 (M⁺+H, C₅₄H₄₈N₆O₆ requires 637.3714).

### EXAMPLE 2

This example illustrates the solid-phase synthesis of Phe-Ach-Phe-Ach-Ile, a peptide analog in accordance with this invention containing three amino acids and two Ach units in alternating positions along the peptide chain.

Merrifield polystyrene resins loaded with Fmoc-amino acids (at approximately 0.7-0.9 mmol/g)were obtained from Calbiochem-NovaBiochem AG (Laufelfingen, Switzerland). Solid phase syntheses were carried out in silylated glass reaction vessels fitted with a frit. The resin was washed in the following manner: DMF (3×), alternating MeOH and CH₂Cl₂ (3× each), and CH₂Cl₂ (3×). When palladium was used in the reaction, the washing included MeOH (1×) prior to the normal washing procedure. During washings, the resin was agitated with nitrogen bubbling for 2 min before the solvent was removed. Presence or absence of free amine was detected by the Kaiser test. Fmoc quantitation analysis was performed with a Uvikon 860 spectrometer (Kontron, Eching, Germany). Reactions were agitated either with a Burrell Wrist Action Shaker (Burrell Scientific, Inc. Pittsburgh, Pa., USA) or a Labquake rotator (Labindustries, Berkeley, Calif., USA). Deprotection of Fmoc was accomplished by shaking the resin in 20% piperidine in DMF for 20 min, followed by the washing procedure and drying of the resin in vacuo for 16-20 h. Resins were stored dry at 0° C.

A. Ach addition

Tin(II) triflate (0.04 g, 0.09 mmol) was added to resin (0.1 g, 0.91 mmol/g) followed by DIEA (0.08 mL, 0.46 mmol), the activated Ach unit prop-2-enyl 3-oxo-5-[(2,4,6-trimethylphenyl)sulfonyloxy]-1,2,6-trihydropyridine-1-carboxylate shown in Example 1 (1 M in CH₂Cl₂, 0.36 mL, 0.36 mmol), and DMF (2.5 mL). The reaction vessel was rotated for 16 h at room temperature, followed by the washing procedure described above and drying of the resin in vacuo for 2 h.

B. Capping

To cap free amines remaining after an acylation or coupling procedure, the resin (0.1 g, 0.91 mmol/g) was prewashed once with dry CH₂Cl₂. The drained resin (0.1 g, 0.91 mmol/g) was swollen in 3:1:1 CH₂Cl₂:DIEA:Ac₂O (5 mL total volume), and the reaction was allowed to proceed for 2 h prior to washing and drying of the resin.

C. Alloc Deprotection

The resin (0.1 g, 0.91 mmol/g) was prewashed once with dry CH₂Cl₂, and then suspended in 3 mL of dry CH₂Cl₂. Me₃SiN(Me)₂ (0.29 mL, 1.8 mmol) was added to the resin followed by Pd(PPh₃)₄ (0.11 g, 0.09 mmol). The resin was quickly shaken for even mixing, followed by rotating for 40 min. The resin was then washed and then dried in vacuo for 2 h.

D. Fmoc-Amino Acid Addition

The resin (0.1 g, 0.91 mmol/g) was prewashed once with dry CH₂Cl₂, then suspended in 3 mL of dry CH₂Cl₂. The desired Fmoc-protected amino acid (5 eq in relation to the resin) was added to the resin, followed by PyBroP (5 eq), and DIEA (10 eq). The reaction vial was vigorously shaken, followed by rotating at room temperature for 24 h. The resin was washed and immediately Fmoc-deprotected by conventional methods.

E. Cleavage From Resin

The product was cleaved from resin immediately after Fmoc-deprotection without drying the resin prior to cleavage. The resin was suspended in 1:1 CH₂Cl₂:TFA (3 mL) and rotated in a glass vial for 2 h. The solvent was removed under reduced pressure, the residue was redissolved in MeOH, filtered and washed (4×2 mL MeOH). This solution was combined and the solvent was removed under reduced pressure; the crude product was immediately purified by preparative HPLC.

F. Solid-Phase Synthesis of Phe-Ach-Ile

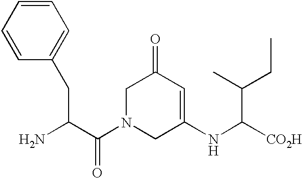

Resin-bound Phe-Ach-Ile (0.71 mmol/g) was assembled from Fmoc-Ile resin according to the general procedures described above. This material (0.46 g resin) was deprotected and cleaved from the resin and purified by preparative reverse-phase HPLC to afford free Phe-Ach-Ile (0.09 g, 0.25 mmol, 75% overall) as a light yellow foam. Although the NMR spectra are complicated due to the presence of rotamers, the structure was confirmed as that shown above by the following: ¹H NMR ((CD₃)₂CO) δ 0.93 (m, 35.9), 1.00 (d, 15.3, J=6.5), 1.04 (d, 1.6, J=7.0), 1.24 (br m, 1.4), 1.34 (m, 7.6), 1.51 (m, 1.9), 1.64 (br m, 7), 1.95 (s, 4.8), 2.06 - 2.07 (m, 47.4), 2.08 (s, 3.8), 3.10 (t, 1.4, J=9.5), 3.21 (m, 6), 3.30 (m, 4.2), 3.38 (m, 5.6), 3.52 (m, 0.46), 3.92 (br m, 17.4), 4.23 (d, 3.9, J=17), 4.33 (m, 1.4), 4.36 (d, 0.4, J=5.5), 4.44 (d, 1, J=6.0), 4.49 (m, 1.6), 4.73 - 4.81 (m, 5.4), 5.06 (m, 3.8), 5.29 (q, 0.3, J=5.0), 5.68 (q, 1.2, J=5.5, J=8.5), 5.82 (q, 1), 6.87 (m, 0.3), 7.30 (m, 28), 7.38 (m, 9), 7.83 (s, 0.2), 7.93 (s, 0.2); ¹³C NMR (CD₃OD) δ 10.19, 10.29, 10.4 (rot), 14.17 (rot), 14.31, 14.59 (rot), 20.89 (rot), 24.87, 25.15, 25.21 (rot), 35.54 (rot), 36.69, 36.79 (rot), 36.88 (rot), 37.05, 41.69, 44.68 (rot), 48.42, 50.86, 50.99 (rot), 51.11 (rot), 60.08 (rot), 60.26, 127.6 (rot), 127.67, 128.45 (rot), 128.64 (rot), 128.94, 129.13 (rot), 129.24, 129.34 (rot), 129.46 (rot), 133.32 (rot), 133.52, 166.81 (rot), 167.01, 171.79 (rot), 171.99, 190.11, 191.37 (rot); IR (film) vₘₐₓ 3264, 2956, 2916, 1672 cm⁻¹; MS (FAB) m/z (%) 374 (M+H⁺, 100), 227 (45), 120 (85); HRMS (FAB) m/z 374.2083 (M+H⁺, C₂₀H₂₈N₃O₄ requires 374.2080).

### G. Solid-Phase Synthesis of Phe-Ach-Phe-Ach-Ile

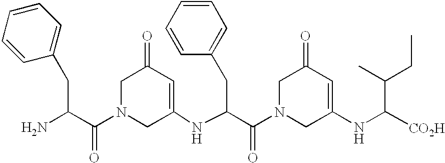

The pentamer Phe-Ach-Phe-Ach-Ile (0.91 mmol/g) was synthesized in a similar manner and a sample of resin (0.06 g) was deprotected and cleaved as described above. The crude material was purified by preparative reverse-phase HPLC to afford the free Phe-Ach-Phe-Ach-Ile (15 mg, 0.03 mmol, 45% overall) as a light yellow foam. While the proton spectrum is complicated due to the presence of rotamers, the structure was confirmed as that shown above by: ¹H NMR (CD₃OD) δ 0.99 (br m, 5.57), 1.31 (br m, 1.6), 1.61 (br m, 0.8), 1.94 (br m, 1), 3.07 (br m, 3.3), 3.86 (br m, 1.5), 4.02 (br m, 0.7), 4.07 - 4.12 (br m, 0.8), 4.22 (br m, 1.0), 4.36 (br m, 0.4), 4.47 (br m, 1), 4.54 (br m, 1), 4.70 (t, 0.5), 4.95 (s, 0.3), 4.99 (d, 0.2), 5.10 (br m, 0.9), 7.17 - 7.31 (br m, 8); IR (film) vₘₐₓ 3318, 2952, 2915, 2847, 1648 cm⁻¹; MS (FAB) m/z (%) 616 (M+H⁺, 70), 340 (60), 312 (90), 284 (100); HRMS (FAB) m/z 616.3118 (M+H⁺, C₃₄H₄₂N₅O₆ requires 616.3135).

### EXAMPLE 3

This example demonstrates the properties of Ach-containing peptide analogs of the present invention, and particularly their unusually high tendency to function as β-strand mimics.

### A. ¹H Spectra as Evidence of Association of Ac-Phe-Ach-Leu-Ach-Ile, N-Methyl Amide as Hydrogen-Bonded Dimer

The self-complementarity of the pentamer synthesized in Example 1 and thus its ability to mimic a β-strand was confirmed by spectral data showing the dimerization of the pentamer and comparing the data with that of a peptide in which the Ach unit is replaced by sarcosine

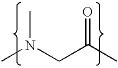

Complete assignments of the ¹³C and ¹H spectra were obtained as follows. Broadband ¹H-decoupled ¹³C spectra were assigned via DEPT subspectra and comparison of observed chemical shifts with those predicted by an NMR simulation program. Two-dimensional HMQC experiments then led directly from the assigned ¹³C chemical shifts to the corresponding ¹H signals. The methylene hydrogens of the two Ach units in Ac-Phe-Ach-Leu-Ach-Ile, N-methyl amide, which show almost identical shifts, could be distinguished by their short-range NOE crosspeaks to nearby aliphatic side chains. Amide hydrogens were assigned from 2D TOCSY spectra acquired in 1% CD₃OH/CDCl₃. The TOCSY spectra also confirmed the other ¹H assignments.

The NH chemical shifts of Ac-Phe-Ach-Leu-Ach-Ile, N-methyl amide in CDCl₃ provided the first indication that this peptide analog forms hydrogen bonds like those of a α sheet. Hydrogen-bonded NH protons in peptides typically resonate around 8 ppm, which is approximately 2 ppm downfield of their chemical shifts when not hydrogen-bonded, per Nowick, J. S., et al., Chem. Soc. Rev. 1996, vol. 25, 401. The δ-values of the NH protons of Ac-Phe-Ach-Leu-Ach-Ile, N-methyl amide are compared with those of the NH protons in the corresponding positions of the sarcosine-containing peptide below, where the protons bear subscripts to show the correspondence between the two formulas:

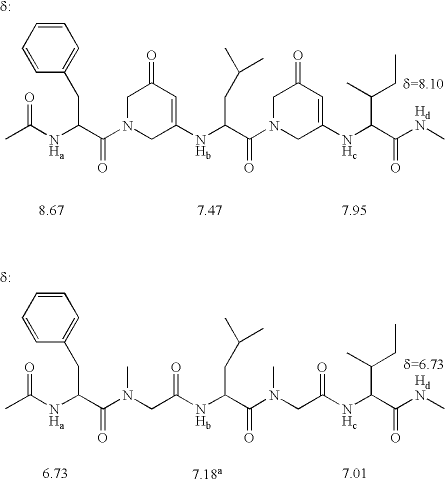

This comparison shows that the NH protons in Ac-Phe-Ach-Leu-Ach-Ile, N-methyl amide, resonate from 7.5-8.7 ppm, which is significantly downfield from the corresponding resonances (6.7-7.2 ppm) observed for the corresponding sarcosine-containing peptide. (The superscript “a” following the δ value for the second NH proton in the sarcosine-containing peptide denotes that the resonance for this amide rotamer was observed at δ 8.12 ppm.) These data indicate that the peptide analog of this invention, Ac-Phe-Ach-Leu-Ach-Ile, N-methyl amide, participates in hydrogen-bonding interactions more extensively than its sarcosine-containing counterpart. However, since two of the NH resonances in Ac-Phe-Ach-Leu-Ach-Ile, N-methyl amide are vinylogous amides, the downfield shifts should be considered in the context of additional evidence supporting a β-sheet model of dimerization.

Some additional evidence is provided by the C_{α}H chemical shifts for Ac-Phe-Ach-Leu-Ach-Ile, N-methyl amide. Relative to the chemical shifts observed for the α-hydrogens of a peptide in an unstructured, random coil conformation, those of an α-helix are shifted upfield and those of a β-strand or extended conformation are downfield, according to Wishart, D. S., et al., Biochemistry 1992, vol. 31, 1647. The chemical shifts for the α-hydrogens of Ac-Phe-Ach-Leu-Ach-Ile, N-methyl amide are shown below, with those expected for the random coil analog (i.e., the sarcosine-containing peptide) in parentheses:

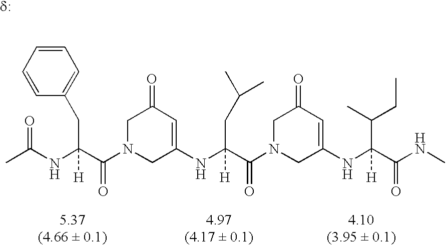

These figures indicate that the chemical shifts for the α-hydrogens of Ac-Phe-Ach-Leu-Ach-Ile, N-methyl amide are well downfield of those expected for a random coil model, which provides further evidence that the Ac-Phe-Ach-Leu-Ach-Ile, N-methyl amide adopts the extended conformation expected in a hydrogen-bonded dimer.

### B. ³J_{HNα} Coupling Constants

The magnitude of the ³J_{HNα} coupling constant for a peptide residue is dependent on the φ-angle and therefore on the local conformation of the polypeptide backbone, according to Smith, L. J., et al., J. Mol. Biol. 1997, vol. 255, 494. ³J_{HNα} values for β-sheet conformations fall in the range from 8 to 10 Hz, while ³J_{HNα} values for an unstructured random coil range from 5.8 to 7.3 Hz. NH-C_{α}H coupling constants for the Leu and Ile residues of Ac-Phe-Ach-Leu-Ach-Ile, N-methyl amide are shown below, where those predicted for a random coil analog (i.e., the corresponding sarcosine-containing peptide) are shown in parentheses:

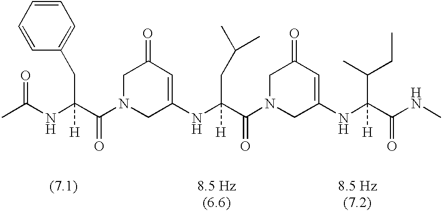

These figures show that the coupling constants for the peptide analog of the invention are within the range for a β-sheet structure and are significantly higher than those of a random coil. Although the differences in coupling constants give an indication of β-sheet conformation for the mimics, they do not provide an indication of the φ-angle directly, since the Karplus equation was derived for peptide amides. Direct comparison of peptide analog of the invention with the sarcosine-containing peptide was not possible, since an NH-C_{α}H coupling constant could only be resolved for the Phe residue in the peptide, which in turn was not resolved for the peptide analog of the invention.

### C. Temperature Dependence of NH Chemical Shifts

Whether an NH group is hydrogen bonded intermolecularly or is exposed to solvent can be revealed by the temperature dependence of the chemical shift (Δδ/ΔT): low values for Δδ/ΔT reflect persistent, intermolecular hydrogen bonds, and high values indicate an equilibrium between hydrogen-bonded and non-bonded states. Values of Δδ/ΔT in 1% CD₃OH/CDCl₃ for both Ac-Phe-Ach-Leu-Ach-Ile, N-methyl amide (as a dimer) and the corresponding sarcosine-containing peptide are shown below.

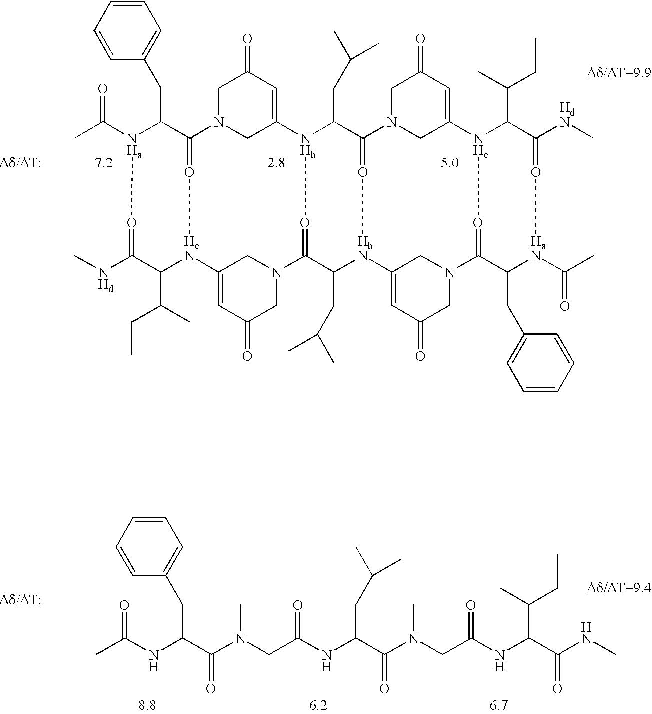

These values show that the Ach-containing peptide analog of the invention exhibits much lower Δδ/ΔT values than its corresponding sarcosine-containing peptide. More revealingly, there are significant differences among the various NH groups of the Ach-containing peptide analog, with those in the center of the strand having lower values than those at the ends. This behavior is consistent with an antiparallel dimer structure in which the NH that is least exposed to the solvent exhibits the smallest Δδ/ΔT value.

### D. Concentration Dependence of NH Chemical Shifts

Dimerization of the Ach-containing peptide analogs of this invention can be detected by observing changes in NH chemical shifts as a function of concentration. For a dimerization process with dissociation constant K_{d} and NMR chemical shifts δₘₒₙₒ and δ_{di}, respectively, the observed chemical shift, δ_{obs}, as a function of concentration c is expressed by the following equation:

$${δ}_{obs} = {δ}_{di} + ( {δ}_{mono} - {δ}_{di} ) \frac{1}{2 c} ( \frac{- {K}_{d}}{2} + \sqrt{\frac{{K}_{d}^{2}}{4} + 2 {K}_{d} c} )$$

Experimental data were obtained for Ac-Phe-Ach-Leu-Ach-Ile, N-methyl amide and for its corresponding sarcosine-containing peptide corresponding to Ac-Phe-Sar-Leu-Sar-Ile, N-methyl amide, as well as for various other Ach-containing peptide analogs of this invention terminating in carboxylic acid groups rather than N-methyl amide groups, all in CDCl₃ or CD₃OH/CDCl₃ at 25° C. The data were fitted to the above equation to give the dissociation constants listed in Table I.

TABLE I
Dissociation Constants (K_{d}) for One Peptide
and Four Ach-Containing Peptide Analogs

| | | Solvent | |
|---|---|---|---|
| | | (% CD₃OH/ | K_{d} |
| Test No. | Peptide or Analog | CDCl₃) | (mM) |
| 1 | Ac-Phe-Sar-Leu-Sar-Ile-NHMe | 0% | >150 |
| 2 | Ac-Phe-Ach-Leu-Ach-Ile-NHMe | 0% | 0.4 |
| 3 | Ac-Leu-Ach-Val-OH | 1% | 35, 71^{a} |
| 4 | Ac-Phe-Ach-Leu-Ach-Val-OH | 2.5% | 0.09 |
| 5 | Ac-Phe-Ach-Leu-Ach-Val-OH | 5% | 8 |
| 6 | Ac-Leu-Ach-Val-Ach-Leu-Ach- | 15%^{b} | 1.5 |
| | Phe-OH | | |

^{a}Amide rotamers with different K_{d} values were observed for Ac-Leu-Ach-Val-OH.
^{b}It was necessary to use 15% CD₃OH in CDCl₃ to observe changes in the chemical shift of this analog with concentration; at lower percentages of CD₃OH, no change was observed down to 0.2 mM.

Table I shows that whereas the dimerization constant determined for the sarcosine-containing peptide (Test No. 1) was greater than 150 mM, the value for its Ach-containing analog (Test No. 2) was 0.4 mM in pure CDCl₃. This demonstrates quantitatively the profound effects that the Ach unit has on the conformation and hydrogen bonding ability of the oligomer. Increasing the length of the oligomer dramatically increases the affinity of the homodimer to such a degree that the dissociation constants for related tri-, penta-, and heptamers (Tests Nos. 3, 4/5, and 6) were not measurable by NMR under the same conditions. Since methanol promotes dissociation, the tri-, penta-, and heptamers were measured at increasing CD₃OH concentrations (as shown in the fourth column of the table). Although direct comparison under identical conditions is not possible, the trend of increasing affinity with increasing oligomer length is quite apparent. It is also noted that the C-terminal carboxylic acid moiety promotes dimerization more strongly than the corresponding N-methyl amide (compare Test No. 2 with Tests Nos. 4 & 5).

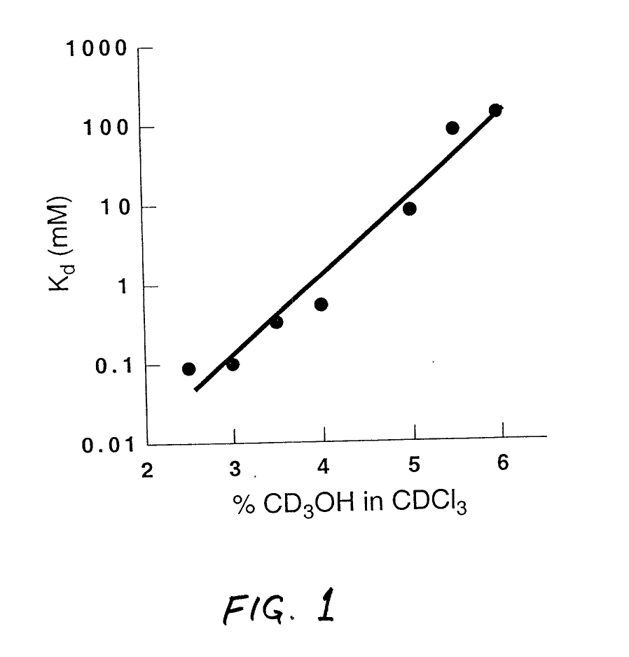

The dissociative effect of methanol was explored with Ac-Phe-Ach-Leu-Ach-Val-OH, and the results are shown in FIG. 1, which is a plot of the dissociation constant K_{d} vs. the methanol (CD₃OH) concentration. The R² for the exponential line fit in this plot is 0.96. The plot shows that the dependence of K_{d} on methanol concentration is dramatic, increasing more than three orders of magnitude between 3% and 6% methanol. The effect is roughly exponential, as would be expected at low concentrations of the dissociating agent, where incremental effects on the free energy of association are additive. Because of sensitivity limitation in the NMR method used to determine the dissociation constants, K_{d} values below 100 μM could not be determined accurately; however, extrapolation of the line in FIG. 1 indicates that the dissociation constant of this peptide analog in pure chloroform could be as low as 0.13 μM.

### E. Nuclear Overhauser Effect Spectroscopy

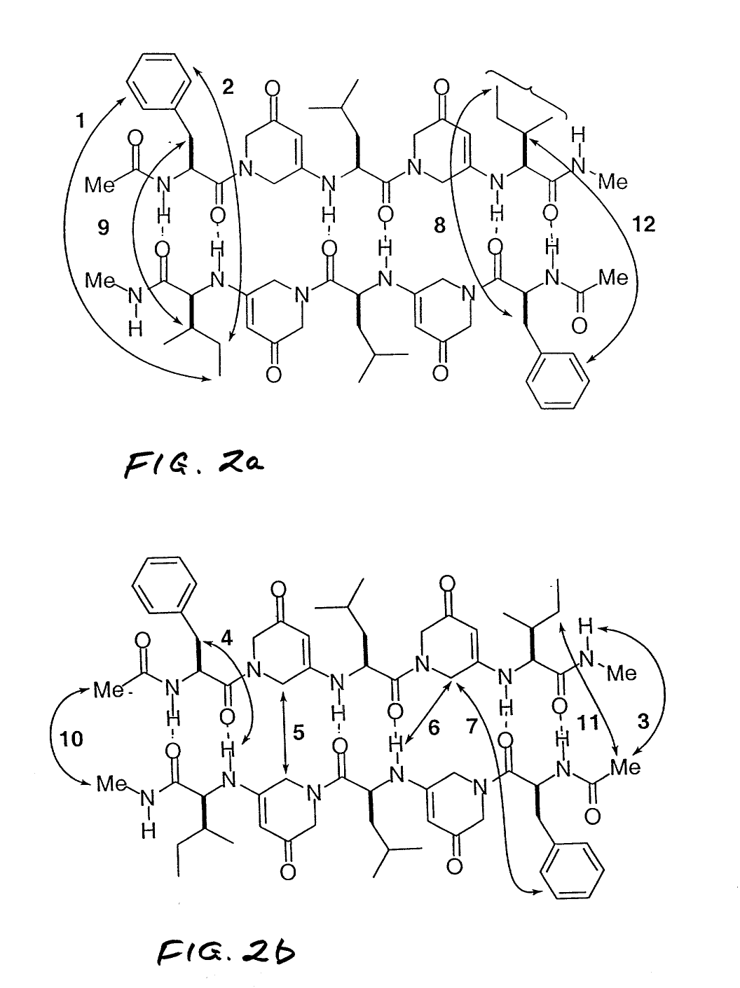

In an antiparallel β-sheet structure, interstrand nuclear Overhauser effects (NOE) are generally observed between the side chains and between the amide hydrogens of opposing residues. Additional evidence for dimer formation of Ach-containing peptide analogs of this invention was thus sought by acquiring NOE spectra of the pentamer Ac-Phe-Ach-Leu-Ach-Ile-NHMe in 1% and 2.5% CD₃OH/CDCl₃, using peptide concentrations of 20-35 mM at 294 K, with mixing times optimized to minimize spin-diffusion. The cross peaks of the spectra are listed in Table II, where the peak intensity is listed as strong (S), medium (M), weak (W), or no peak observed (N/O). The NOE interactions are also identified in FIG. 2.

TABLE II
Intermolecular NOE Crosspeaks Observed for
Ac-Phe-Ach-Leu-Ach-Ile-NHMe in CD₃OH/CDCl₃
Using Two Concentrations of CD₃OH

| NOE | | | Crosspeak Strength | |
|---|---|---|---|---|
| No. | Protons Involved | CD₃OH Conc. → | 1% | 2.5% |
| 1 | Phe-aryl-Ile-δ | | S | W |
| 2 | Phe-aryl-Ile-γ | | W | N/O |
| 3 | Ac-Me-NH-CH₃ | | M | N/O |
| 4 | Phe-β-Ile-NH | | W | W |
| 5 | Ach-I-γ-Ach-II-γ | | M | M |
| 6 | Ach-II-γ-Leu-NH | | W | N/O |
| 7 | Ach-II-γ-Phe-aryl | | W | N/O |
| 8 | Ile-δ-Phe-β | | S | N/O |
| 9 | Ile-β-Phe-β | | S | M |
| 10 | NH—CH₃-Ac | | S | M |
| 11 | Ile-δ-Ac | | S | N/O |
| 12 | Ile-β-Phe-aryl | | M | N/O |

These data show that at a concentration of 20 mM, the spectrum of the pentamer Ac-Phe-Ach-Ile-NHMe shows crosspeaks between hydrogens at opposite ends of the molecule, which would not be expected to arise intramolecularly. Spectra obtained at increasing CD₃OH concentrations demonstrated that these crosspeaks are intermolecular; they are weaker in 2.5% CD₃OH/CDCl₃ and absent entirely in 10% CD₃OH/CDCl₃. For comparison, the NOE spectrum for the corresponding sarcosine-containing peptide, which was obtained using the same parameters, solvent, and concentration as those for the Ach-containing analog, showed no crosspeaks between hydrogens at opposite ends of the molecule.

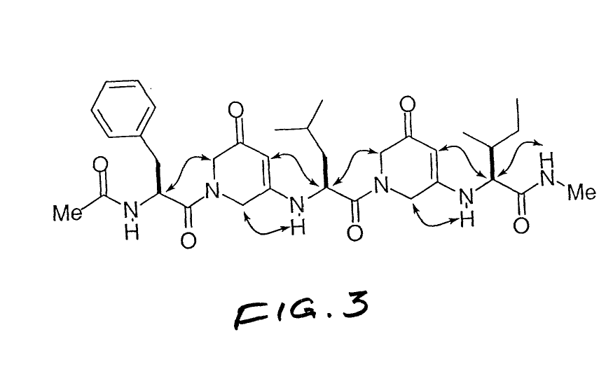

Further evidence for the β-strand conformation of Ac-Phe-Ach-Leu-Ach-Ile-NHMe is provided by the intramolecular crosspeaks in the NOE spectrum. These are shown in FIG. 3. The crosspeaks between the C_{α} hydrogens of the amino acids and the C2 methylene and C4 vinyl hydrogens of the Ach units were consistent with a conformation in which these atoms lie close to each other in the pleated conformation. Similarly, crosspeaks were observed between the C6 methylene hydrogens of the Ach units and the Leu and Ile amide hydrogens. Equally telling are the crosspeaks that are not observed, for example between the amide hydrogens and the C2 and C4 positions, or the C_{α} hydrogens and the C6 position.

The experimental data presented above demonstrate that replacing amino acids at alternate positions in a peptide with the 1,2-dihydro-3(6B)-pyridinone (“Ach”) unit affords an oligomeric molecule that shows many of the NMR and hydrogen-bonding characteristics of a peptide in the extended, β-strand conformation in chloroform and chloroform/methanol. This behavior is revealed by an enhanced propensity to dimerize in comparison to a related peptide, by reduced exposure of the central NH groups to solvent, and by a pattern of solvent-dependent NOE interactions that are consistent with an antiparallel hydrogen bonded dimer. This indicates that the peptide analogs of the present invention are unusually effective as β-strand mimetics and are useful in physiological processes involving β-sheet formation.

### EXAMPLE 4

This example demonstrates the properties of hybrids of peptides and Ach-containing peptide analogs that form intramolecular anti-parallel β-sheets, i.e., covalently linked chains consisting of a peptide segment linked to an Ach-containing segment through a β-turn (commonly known as a “hair-pin”) linkage. The sharp turn of the linkage places the peptide and Ach-containing segments in a conformation that permits them to engage in a β-sheet-like interaction, which is stabilized both by the Ach units and by the covalent linkage between the two segments. This dimerization renders the peptide segment a particularly strong complexing agent for β-sheet-like interactions with other peptides.

The D-proline-alanine sequence was used as the linkage group, and using the general solid-phase synthesis procedures described in the preceding examples, the peptide Phe-Gly-Ser-D-Pro-Ala-Leu-Ach-Ile and its counterpart in which the Ach group is replaced by sarcosine were prepared. The structures of these products, identified herein as products 1 and 2, respectively, each shown in β-sheet conformation, are as follows:

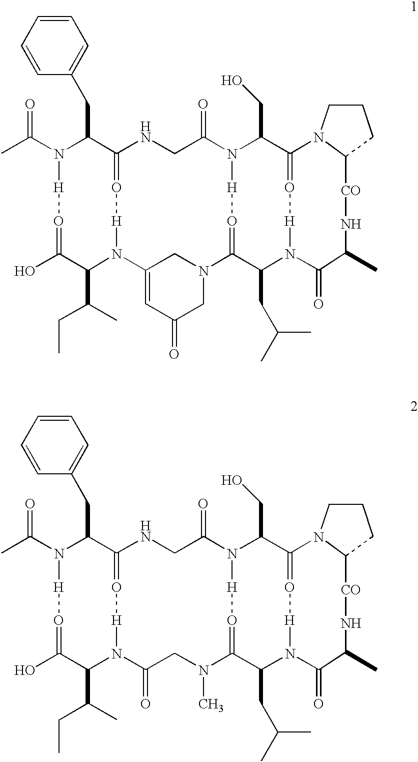

Evidence to confirm that the hybrid 1 assumed a β-sheet structure was obtained using NMR techniques. Proton resonance assignments were made in a sequence specific manner by the method of Wüthrich, K., NMR of Proteins and Nucleic Acids, John Wiley & Sons: New York, 1986. Thus, individual spin systems were identified using COSY connectivities within a residue and sequential NOESY connectivities, dαN(i, i+1) between adjacent residues. The individual spin systems and amide protons for each residue were assigned using TOCSY spectra and verified by NOE correlations.

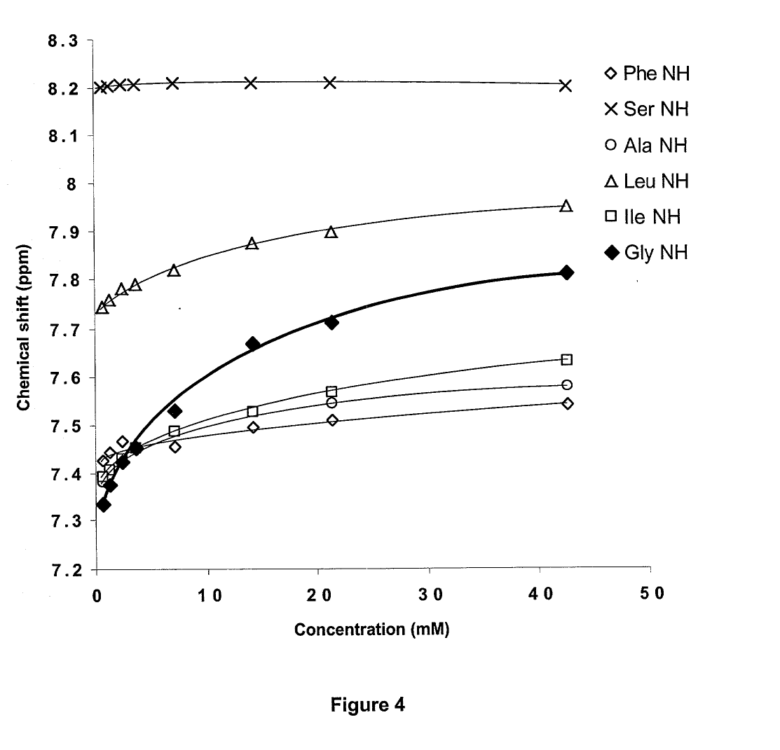

To confirm that the species being analyzed were predominantly monomeric β-turn sequences, the concentration-dependencies of the amide proton chemical shifts were obtained for both the hybrid 1 and it sarcosine analog 2 over the concentration range 0.6-42 mM. The hybrid 1 shows the greatest concentration dependency for the NH of the glycine residue (see FIG. 4). To distinguish between the two species, the dissociation constants of dimers of each species were determined. The hybrid 1 was found to have a dimer dissociation constant of 25 mM, while the dimer dissociation constant of the sarcosine analog 2 was found to be >300 mM, both in 5% CD₃OH/CDCl₃. This behavior is consistent with formation of a dimeric complex in which only the glycine NH is available for intermolecular hydrogen bonding across a dimer interface, i.e., all other CO and NH groups are engaged in intramolecular hydrogen bonding between the two segments of the chain. This dimeric complex is as follows:

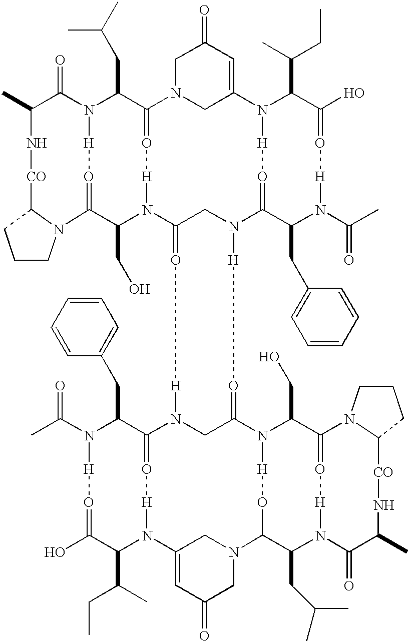

### Templating Ability of Hybrid 1 as Shown by NMR Evidence of β-Sheet Formation

The NH chemical shifts of the hybrid 1 in 5% CD₃OH/CDCl₃ resonate significantly downfield of their positions in a non-hydrogen-bonded peptide. This behavior is consistent with the existence of intramolecular hydrogen-bonding interactions in the structure of 1. Also, the CαH chemical shifts for the hybrid are downfield of those for a peptide in an unstructured conformation. This behavior is evidence for a β-strand or extended conformation. The ³J_{HNα} coupling constants, which are sensitive to the conformation of a peptide backbone, are a further indication. Those for the hybrid 1 were within the range expected for a β-sheet structure. Moreover, the amide protons of the hybrid demonstrated a smaller temperature dependence of the chemical shift than observed for the corresponding sarcosine analog 2, which is further evidence for an intramolecularly hydrogen-bonded conformation.

Intramolecular NOE effects provide detailed information on the conformation of a peptide. The hybrid 1 and its sarcosine analog 2 were therefore both analyzed in this manner. The results for the hybrid 1 showed a large number of inter-chain NOEs, which is consistent with the folded structure of a templated β-sheet, compared to fewer NOEs for the sarcosine analog 2 under similar conditions. Solvent effect and concentration studies were also performed, providing further evidence of the dimerization of the folded hybrid 1, which is further evidence of the ability of the Ach-containing segment of the hybrid to induce the peptide segment to adopt an extended conformation.

Finally, the CD spectra for the hybrid 1 and its sarcosine analog 2 were taken in CF₃CH₂OH. The spectrum for the hybrid was consistent with a β-turn conformation, exhibiting a maximum at approximately 198 nm and a minimum at approximately 230 nm. The sarcosine analog 2 exhibited a maximum at approximately 200 nm but no apparent minimum in the neighborhood of 230 nm. These studies confirm that the hybrid 1 is significantly more effective than its sarcosine analog 2 in stabilizing the linked peptide segment in a β-sheet structure.

### Templating Ability of Hybrid 1 in Aqueous Solution

It is known that a peptide in β-sheet conformation exhibits a maximum in a CD spectrum of about 195 nm and a minimum of about 217 nm. A β-turn gives a maximum of about 205 nm and a minimum of about 220 nm, although these values are dependent on the type of turn. For a random coil conformation, the signal below 210 nm is increasingly negative.

To confirm the ability of the Ach-containing hybrids to stabilize a peptide in the extended or β-strand conformation, CD spectra were taken on several hybrids that varied in length, relative position, and side chain structure, as well as the sacrosine analog of one of these hybrids and a hybrid with an L-proline-alanine linkage rather than a D-proline-alanine linkage. The structures of these compounds are shown below (where not shown explicitly, the amino acid side chains in the structures of 3 to 7 are designated by their three-letter codes):

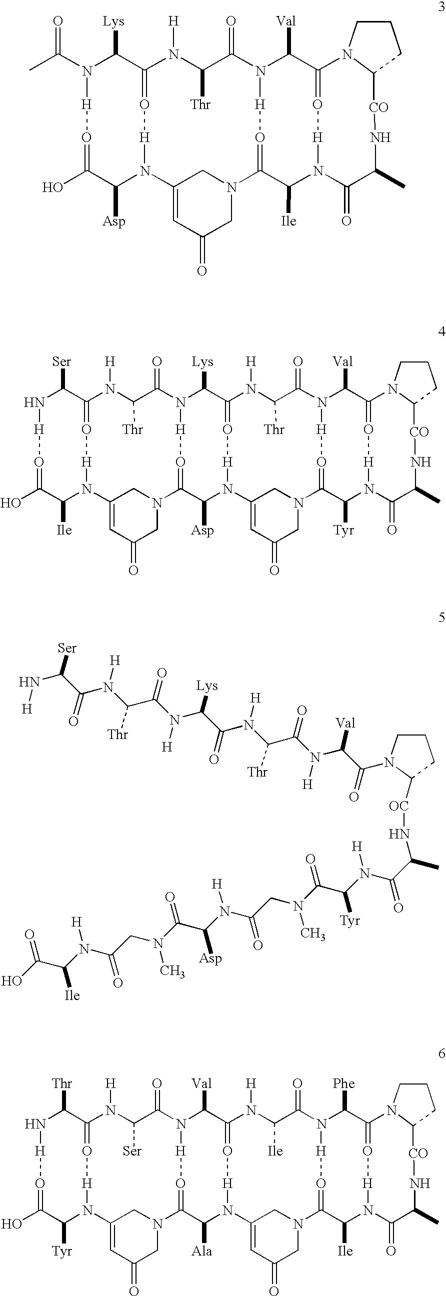

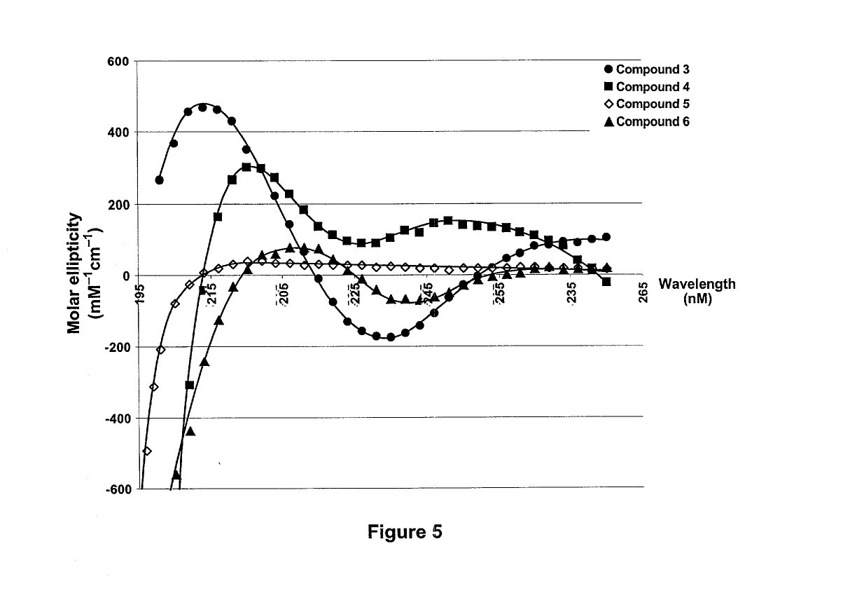

These compounds were analyzed by CD for their ability to form a β-sheet-β-turn structure in aqueous solutions (0.1 mM sodium phosphate pH 7). The results are shown in FIG. 5. A comparison of the Ach-containing species 4 with its sarcosine analog 5 shows that the Ach-containing species significantly stabilizes the hairpin β-sheet-β-turn structure shown above. In the CD spectrum for 4 there is a maximum around 210 nm and a minimum around 226 nm, both of which are consistent with a β-sheet plus β-turn structure. No such maximum or minimum appear in the CD spectrum of the sarcosine analog 5. Even compound 3, which has only a single Ach unit, is capable of adopting a β-turn, thus stabilizing the peptide segment in a β-sheet conformation in water.

The analog 7 that included an L-proline in the linkage rather than a D-proline did not display a β-turn CD signature. The signature that this analog did display was that of an unstructured peptide, in clear contrast to the other analogs.

### β-Turn-Promoting Linkage Other than D-Pro-Ala

An alternative to the D-proline-alanine linkage is the asparagine-glycine linkage, and a hybrid containing this linkage was used in preparing the following structure:

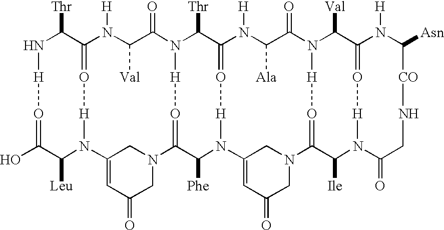

The CD spectrum of this structure in the aqueous solution described above was consistent with a β-turn structure. Spectra were taken up to 70° C. with no significant changes observed, indicating that the folded structure is stable at those elevated temperatures.

The foregoing is offered primarily for purposes of illustration. Further modifications and variations that still embody the underlying concepts of the invention and fall within its scope will be apparent to those skilled in the art.

What is claimed is:

1. A compound having the formula 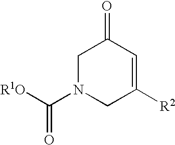
in which:
R¹ is a protecting group other than methyl and ethyl; and
R² is a member selected from the group consisting of OH and activated leaving groups.

2. A compound in accordance with claim 1 in which R² is OH.

3. A compound in accordance with claim 1 in which R² is an activated leaving group.

4. A compound having a formula selected from the group consisting of 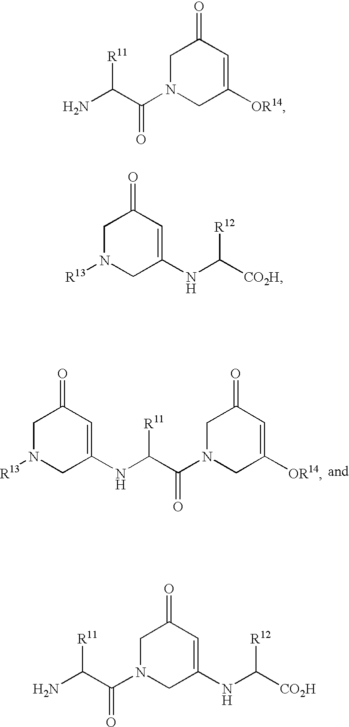
in which:
R¹¹ is an amino acid side chain;
R¹² is an amino acid side chain;
R¹³ is a member selected from the group consisting of H and amine protecting groups; and
R¹⁴ is a member selected from the group consisting of H and carboxy protecting groups;
and amine-protected analogs of those of said group that terminate in H₂N—, carboxy-protected analogs of those of said group that terminate in —CO₂H, carboxy-activated analogs of those of said group that terminate in —CO₂H, amine-protected and carboxy-protected analogs of 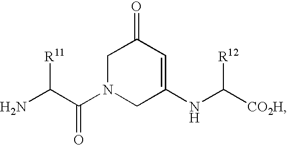
and amine-protected and carboxy-activated analogs of

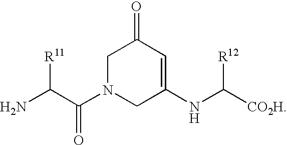

5. A compound in accordance with claim 4 which is a member selected from the group consisting of compounds of the formula 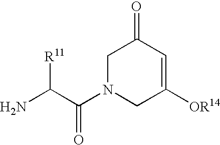
in which R¹⁴ is a carboxy protecting group, and amine-protected analogs of said compounds.

6. A compound in accordance with claim 4 which is a member selected from the group consisting of compounds of the formula 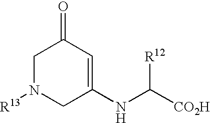
in which R¹³ is an amine protecting group, and carboxy-protected analogs of said compounds.

7. A compound in accordance with claim 4 which is a member selected from the group consisting of compounds of the formula 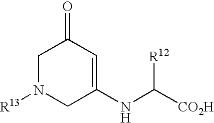
in which R¹³ is an amine protecting group, and carboxy-activated analogs of said compounds.

8. A compound in accordance with claim 4 which is a member selected from the group consisting of compounds of the formula 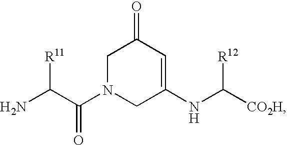
amine-protected analogs thereof, carboxy-protected analogs of said compounds, carboxy-activated analogs of said compounds; amine-protected and carboxy-protected analogs of said compounds, and amine-protected and carboxy-activated analogs of said compounds.

9. A compound in accordance with claim 4 in which R¹¹ and R¹² are side chains of natural amino acids.

10. A compound in accordance with claim 4 in which at least one of R¹¹ and R¹² is a side chain of an unnatural amino acid.

11. A peptide analog comprising a peptide in which at least one amino acid, but less than all amino acids, is replaced by an azacyclohexenone group having the formula

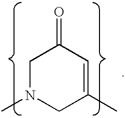

12. A peptide analog in accordance with claim 11 in which the amino acids of said peptide analog are from 2 to 200 in number and the azacyclohexenone groups are from 1 to 100 in number.

13. A peptide analog in accordance with claim 12 in which the number ratio of said azacyclohexenone groups to amino acids is from 1:10 to 10:1.

14. A peptide analog in accordance with claim 11 in which the amino acids of said peptide analog are from 2 to 100 in number and the azacyclohexenone groups are from 1 to 50 in number.

15. A peptide analog in accordance with claim 14 in which the number ratio of said azacyclohexenone groups to amino acids is from 1:10 to 10:1.

16. A peptide analog in accordance with claim 11 in which the amino acids of said peptide analog are from 2 to 20 in number and the azacyclohexenone groups are from 1 to 20 in number.

17. A peptide analog in accordance with claim 11 in which the amino acids of said peptide analog are from 2 to 10 in number and the azacyclohexenone groups are from 1 to 20 in number.

18. A peptide analog in accordance with claim 11 in which the number ratio of said azacyclohexenone groups to amino acids is from 1:5 to 5:1.

19. A peptide analog in accordance with claim 11 in which the number ratio of said azacyclohexenone groups to amino acids is from 1:2 to 1:1.

20. A peptide analog having the formula 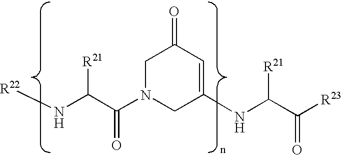
in which:
the R²¹'s are the same or different and each R²¹ is an amino acid side chain;
R²² is a member selected from the group consisting of peptide chain terminating groups and 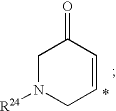
in which R²⁴ is a member selected from the group consisting of H, alkyl, acyl, carbamoyl, and alkoxycarbonyl, and * denotes the site of attachment;
R²³ is a member selected from the group consisting of peptide chain terminating groups and 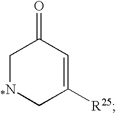
in which R²⁵ is a member selected from the group consisting of hydroxyl, alkoxy, alkylamino, dialkylamino, and arylamino, and * denotes the site of attachment; and
n is at least 2.

21. A peptide analog in accordance with claim 20 in which the R²¹'s are a combination of side chains of natural and unnatural amino acids.

22. A peptide analog in accordance with claim 20 in which the R²¹'s are side chains of natural amino acids.

23. A peptide analog in accordance with claim 20 in which R²² is a member selected from the group consisting of acyl, carbamoyl, and alkoxycarbonyl.

24. A peptide analog in accordance with claim 20 in which R²² is acetyl.

25. A peptide analog in accordance with claim 20 in which R²² is

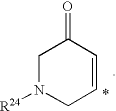

26. A peptide analog in accordance with claim 20 in which R²³ is a member selected from the group consisting of hydroxyl, alkoxy, alkylamino, dialkylamino, and arylamino.

27. A peptide analog in accordance with claim 20 in which R²³ is a member selected from the group consisting of hydroxyl and methylamino.

28. A peptide analog in accordance with claim 20 in which R²³ is

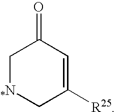

29. A peptide analog in accordance with claim 20 in which n is 2 to 100.

30. A peptide analog in accordance with claim 20 in which n is 2 to 50.

31. A peptide analog in accordance with claim 20 in which n is 2 to 5.

32. A peptide analog comprising a first segment consisting of a sequence of amino acids joined by amide bonds and a second segment consisting of a sequence of amino acids joined by amide bonds, in which at least one amino acid, but less than all amino acids, of said second segment is replaced by an azacyclohexenone group having the formula 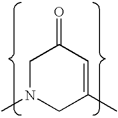
said first and second segments joined by a covalent linkage that permits said first and second segments to adopt a β-sheet-like interaction.

33. A peptide analog in accordance with claim 32 in which said second segment consists of an amino acid sequence in which two or more non-adjacent amino acids are replaced by azacyclohexenone groups of said formula.

34. A peptide analog in accordance with claim 32 in which, in at least a portion of said second segment, every second amino acid is replaced by an azacyclohexenone group of said formula.

35. A peptide analog in accordance with claim 32 in which said first segment contains from 3 to 200 amino acids and in said second segment the total number of amino acids and azacyclohexenone groups is from 3 to 200.

36. A peptide analog in accordance with claim 32 in which said first segment contains from 3 to 100 amino acids and in said second segment the total number of amino acids and azacyclohexenone groups is from 3 to 100.

37. A peptide analog in accordance with claim 32 in which said first segment contains from 3 to 20 amino acids and in said second segment the total number of amino acids and azacyclohexenone groups is from 3 to 20.

38. A peptide analog in accordance with claim 32 in which said covalent linkage is a member selected from the group consisting of D-Pro-Ala and Asn-Gly.

39. A method for inhibiting the association of a selected peptide with other peptides, said method comprising contacting said selected peptide with a peptide analog defined as a peptide in which at least one amino acid, but less than all amino acids, is replaced by an azacyclohexenone group having the formula 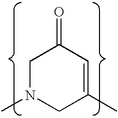
to achieve a β-sheet-like interaction between said peptide and said peptide analog.

40. A method in accordance with claim 39 in which said peptide analog is a peptide in which two or more non-adjacent amino acids are replaced by azacyclohexenone groups of said formula.

41. A method in accordance with claim 39 in which said peptide analog is a peptide in which, in at least a portion thereof, every second amino acid is replaced by an azacyclohexenone group of said formula, and the number of said azacyclohexenone groups in said peptide analog is two or more.

42. A method in accordance with claim 39 in which said peptide analog is a peptide in which every second amino acid is replaced by an azacyclohexenone group of said formula.

43. A method in accordance with claim 39 in which the total number of amino acids and azacyclohexenone groups in said peptide analog is from 3 to 200.

44. A method in accordance with claim 39 in which the total number of amino acids and azacyclohexenone groups in said peptide analog is from 3 to 100.

45. A method in accordance with claim 39 in which the total number of amino acids and azacyclohexenone groups in said peptide analog is from 4 to 20.

46. A method in accordance with claim 39 in which the total number of amino acids and azacyclohexenone groups in said peptide analog is from 4 to 10.

47. A method for inhibiting the association of a peptide with a double-stranded nucleic acid, said method comprising contacting said peptide with a peptide analog defined as a peptide in which at least one amino acid, but less than all amino acids, is replaced by an azacyclohexenone group having the formula 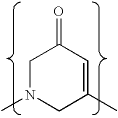
to achieve a β-sheet-like interaction between said peptide and said peptide analog.

48. A method in accordance with claim 47 in which said peptide analog is a peptide in which two or more non-adjacent amino acids are replaced by azacyclohexenone groups of said formula.

49. A method in accordance with claim 47 in which said peptide analog is a peptide in which, in at least a portion thereof, every second amino acid is replaced by an azacyclohexenone group of said formula, and the number of said azacyclohexenone groups in said peptide analog is two or more.

50. A method in accordance with claim 47 in which said peptide analog is a peptide in which every second amino acid is replaced by an azacyclohexenone group of said formula.

51. A method in accordance with claim 47 in which the total number of amino acids and azacyclohexenone groups in said peptide analog is from 3 to 200.

52. A method in accordance with claim 47 in which the total number of amino acids and azacyclohexenone groups in said peptide analog is from 3 to 100.

53. A method in accordance with claim 47 in which the total number of amino acids and azacyclohexenone groups in said peptide analog is from 4 to 20.

54. A method in accordance with claim 47 in which the total number of amino acids and azacyclohexenone groups in said peptide analog is from 4 to 10.

55. A method for inhibiting the biological activity of a peptide, said method comprising contacting said peptide with a peptide analog defined as a peptide in which at least one amino acid, but less than all amino acids, is replaced by an azacyclohexenone group having the formula 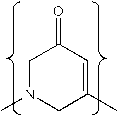
to achieve a β-sheet-like interaction between said peptide and said peptide analog.

56. A method in accordance with claim 55 in which said peptide analog is a peptide in which two or more non-adjacent amino acids are replaced by azacyclohexenone groups of said formula.

57. A method in accordance with claim 55 in which said peptide analog is a peptide in which, in at least a portion thereof, every second amino acid is replaced by an azacyclohexenone group of said formula, and the number of said azacyclohexenone groups in said peptide analog is two or more.

58. A method in accordance with claim 55 in which said peptide analog is a peptide in which every second amino acid is replaced by an azacyclohexenone group of said formula.

59. A method in accordance with claim 55 in which the total number of amino acids and azacyclohexenone groups in said peptide analog is from 3 to 200.

60. A method in accordance with claim 55 in which the total number of amino acids and azacyclohexenone groups in said peptide analog is from 3 to 100.

61. A method in accordance with claim 55 in which the total number of amino acids and azacyclohexenone groups in said peptide analog is from 4 to 20.

62. A method in accordance with claim 55 in which the total number of amino acids and azacyclohexenone groups in said peptide analog is from 4 to 10.

63. A method for increasing the tendency of a target peptide or a portion of a target peptide to assume a β-strand conformation, said method comprising contacting said target peptide with a peptide analog defined as a peptide in which at least one amino acid, but less than all amino acids, is replaced by an azacyclohexenone group having the formula 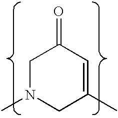
to achieve a β-sheet-like interaction between said target peptide and said peptide analog.

64. A method in accordance with claim 63 in which said peptide analog is a peptide in which two or more non-adjacent amino acids are replaced by azacyclohexenone groups of said formula.

65. A method in accordance with claim 63 in which said peptide analog is a peptide in which, in at least a portion thereof, every second amino acid is replaced by an azacyclohexenone group of said formula, and the number of said azacyclohexenone groups in said peptide analog is two or more.

66. A method in accordance with claim 63 in which said peptide analog is a peptide in which every second amino acid is replaced by an azacyclohexenone group of said formula.

67. A method in accordance with claim 63 in which the total number of amino acids and azacyclohexenone groups in said peptide analog is from 3 to 200.

68. A method in accordance with claim 63 in which the total number of amino acids and azacyclohexenone groups in said peptide analog is from 4 to 20.

69. A method for extracting a target peptide having a selected amino acid sequence from a mixture of peptides, said method comprising contacting said mixture with a capture peptide that is covalently bonded to a solid support and associates with said amino acid sequence in a β-sheet interaction, said capture peptide comprising amino acids and at least one azacyclohexenone group having the formula

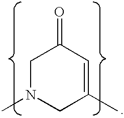

70. A method in accordance with claim 69 in which said capture peptide comprises amino acids and two or more non-adjacent azacyclohexenone groups of said formula.

71. A method in accordance with claim 69 in which at least a portion of said capture peptide comprises amino acids alternating with azacyclohexenone groups of said formula, and the number of said azacyclohexenone groups in said capture peptide is two or more.

72. A method in accordance with claim 69 in which said capture peptide consists of amino acids alternating with azacyclohexenone groups of said formula.

73. A method in accordance with claim 69 in which the total number of amino acids and azacyclohexenone groups in said capture peptide is from 3 to 200.

74. A method in accordance with claim 69 in which the total number of amino acids and azacyclohexenone groups in said capture peptide is from 4 to 20.
